(19) United States
(12) Patent Application Publication
(10) Pub. No.: US 2022/0395559 A1
(43) Pub. Date: Dec. 15, 2022

(54) SOLID COMPOSITIONS COMPRISING A GLP-1 AGONIST, AN SGLT2 INHIBITOR AND A SALT OF N-(8-(2-HYDROXYBENZOYL)AMINO)CAPRYLIC ACID

(71) Applicant: Novo Nordisk A/S, Bagsvaerd (DK)

(72) Inventors: Kaisa Naelapaeae, Havdrup (DK); Simon Bjerregaard, Hilleroed (DK); Ulrik Lytt Rahbek, Charlottenlund (DK); Philip Jonas Sassene, Copenhagen V (DK); Jorrit Jeroen Water, Frederiksberg (DK); Andreas Vegge, Frederiksberg (DK)

(21) Appl. No.: 17/775,034

(22) PCT Filed: Nov. 6, 2020
(86) PCT No.: PCT/EP2020/081231

§ 371 Date: May 6, 2022

(30) Foreign Application Priority Data
Nov. 7, 2019 (EP) 19207737.8
Apr. 30, 2020 (EP) 20172419.2

(51) Int. Cl. A61K 38/26; A61K 31/166; A61K 31/455; A61K 31/05; A61K 45/06; A61K 31/7048; A61K 9/20

(52) U.S. Cl. CPC A61K 38/26; A61K 31/166; A61K 31/455; A61K 31/05; A61K 45/06; A61K 31/7048; A61K 9/2013; A61K 9/2077; A61K 9/2095

(57) ABSTRACT

The invention relates to pharmaceutical compositions comprising a GLP-1 agonist, an SLGT2 inhibitor, a salt of N-(8-(2-hydroxybenzoyl)amino)caprylic acid, and a hydrotrope. The invention further relates to processes for the preparation of such compositions, and their use in medicine.

### TECHNICAL FIELD OF THE INVENTION

The present invention relates to solid compositions comprising a GLP-1 agonist, an SGLT2 inhibitor and a salt of N-(8-(2-hydroxybenzoyl)amino)caprylic acid, their method of preparation and their use in medicine.

### INCORPORATION-BY-REFERENCE OF THE SEQUENCE LISTING

The Sequence Listing, entitled “SEQUENCE LISTING”, is 2 KB and was created 4 Nov. 2020 and is incorporated herein by reference.

### BACKGROUND

Human GLP-1 and analogues thereof have a low oral bioavailability. Exposure and bioavailability of human GLP-1 and analogues thereof is very low following oral administration. Human GLP-1 and analogues thereof can only reach therapeutically relevant plasma concentration after oral administration if formulated with certain absorption enhancers in a specific amount.

Steinert et al. (Am J Clin Nutr, October 2010; 92: 810-817) discloses oral administration of a tablet comprising GLP-1(7-36)amide and 150 mg sodium N-(8-(2-hydroxybenzoyl)amino)caprylate (SNAC).

WO2010/020978 discloses an oral pharmaceutical composition comprising a protein and N-(8-(2-hydroxybenzoyl)amino)caprylate (NAC). Patent applications disclosing oral dosage forms of GLP-1 analogues containing a salt of N-(8-(2-hydroxybenzoyl)amino)caprylate include WO2012/080471, WO2013/189988, WO2013/139694, WO2013/139695 and WO2014/177683.

Despite these findings there is still room for a further improved pharmaceutical compositions for oral administration of a GLP-1 agonist, such as semaglutide.

### SUMMARY

The present invention relates to a composition comprising a GLP-1 agonist, an SGLT2 inhibitor, an absorption enhancer or delivery agent and a hydrotrope. The composition according to the invention comprises balanced amounts of the delivery agent and the hydrotrope. The provided compositions display an accelerated absorption, enabling fast and efficient uptake of the active pharmaceutical ingredient.

Oral administration of therapeutic peptides is challenging, due to the rapid degradation of such peptides in the gastrointestinal system.

Described herein are pharmaceutical compositions providing accelerated absorption of the GLP-1 agonist within 15-30 minutes after administration and thereby improved exposure of the GLP-1 agonist by oral administration. The inventors have surprisingly found that an increased exposure of GLP-1 agonists is observed when compositions are prepared with a hydrotrope.

In some embodiments the present invention relates to a composition comprising a GLP-1 agonist (such as semaglutide), 1-50 mg SGLT2 inhibitor, a salt of N-(8-(2-hydroxybenzoyl)amino)caprylic acid (NAC), and a hydrotrope, wherein the hydrotrope is capable of increasing the solubility of SNAC at least 2-fold, such as 5-fold or such as at least 10-fold.

In some embodiments the composition comprises: 0.1-50 mg GLP-1 agonist (such as semaglutide), 1-50 mg SGLT2 inhibitor, 50-600 mg salt of NAC (such as the sodium salt of NAC (SNAC)), 20-400 mg nicotinamide or resorcinol, and 0-20 mg lubricant.

The present invention also relates to a method for producing a solid pharmaceutical composition comprising the steps of: i) co-processing a salt of NAC and a hydrotrope (optionally with an SGLT2 inhibitor), and ii) preparing said solid pharmaceutical composition using the product of i), wherein said solid pharmaceutical composition comprises a GLP-1 agonist and an SGLT2 inhibitor.

In some embodiments the invention relates to the medical use of compositions described herein, such as for oral administration and/or for use in treating diabetes and/or obesity.

### BRIEF DESCRIPTION OF DRAWINGS

FIG. 1 shows dose dependent effect of nicotinamide (A) and resorcinol (B) on SNAC solubility at pH 6.

FIG. 2 shows dissolution of reference and test compositions.

### DESCRIPTION

Aspects of the invention described herein relate to a composition comprising a GLP-1 agonist, an SGLT2 inhibitor, an absorption enhancer or delivery agent and a hydrotrope. The composition may be in the form suitable for oral administration, such as in a solid form exemplified by a tablet, sachet or capsule. In some embodiments the composition is an oral composition, or a pharmaceutical composition, such as an oral pharmaceutical composition. The provided compositions display an accelerated absorption, enabling fast and efficient uptake of the active pharmaceutical ingredient.

In some embodiments the present invention relates to a composition comprising a GLP-1 agonist, 1-50 mg SGLT2 inhibitor, a salt of NAC, and a hydrotrope, wherein the hydrotrope is capable of increasing the solubility of SNAC at least 2-fold, such as 5-fold or such as at least 10-fold.

In some embodiments the composition comprises: 0.1-50 mg GLP-1 agonist, 1-50 mg SGLT2 inhibitor, 50-600 mg salt of NAC (such as the sodium salt of NAC (SNAC)), 20-400 mg nicotinamide or resorcinol, and 0-20 mg lubricant.

The present invention also relates to a method for producing a solid pharmaceutical composition comprising the steps of: i) co-processing a salt of NAC and a hydrotrope (optionally with an SGLT2 inhibitor), and ii) preparing said solid pharmaceutical composition using the product of i), wherein said solid pharmaceutical composition comprises a GLP-1 agonist and an SGLT2 inhibitor.

The present invention also relates to a method for producing a solid pharmaceutical composition comprising the steps of: i) obtaining a blend comprising a salt of NAC and a hydrotrope (optionally with an SGLT2 inhibitor), ii) co-processing the blend of i), and iii) preparing said solid pharmaceutical composition using the product of ii), wherein said solid pharmaceutical composition also comprises a GLP-1 agonist and an SGLT2 inhibitor.

In some embodiments the invention relates to the medical use of compositions described herein, such as for oral administration and/or for use in treating diabetes and/or obesity.

### GLP-1

In some embodiments the compositions of the invention comprise a GLP-1 agonist. The term “GLP-1 agonist” as used herein refers to a compound, which fully or partially activates the human GLP-1 receptor. In some embodiments, the GLP-1 agonist for use in the present invention is an acylated GLP-1 agonist. The term “acylated” as used in relation to GLP-1 agonists refers to the GLP-1 agonist having covalently attached at least one substituent comprising a lipophilic moiety, such as a fatty acid or a fatty diacid. In some embodiments the substituent comprises a fatty acid or a fatty diacid. In some embodiments, the term GLP-1 agonist as well as the specific GLP-1 agonists described herein also encompass salt forms thereof.

It follows that the GLP-1 agonist should display “GLP-1 activity” which refers to the ability of the compound, i.e. GLP-1 agonist, to bind to the GLP-1 receptor and initiate a signal transduction pathway resulting in insulinotropic action or other physiological effects as is known in the art. In some embodiments the “GLP-1 agonist” binds to a GLP-1 receptor, e.g., with an affinity constant (K_{D}) or activate the receptor with a potency (EC₅₀) of below 1 μM, e.g. below 100 nM as measured by methods known in the art (see e.g. WO 98/08871) and exhibits insulinotropic activity, where insulinotropic activity may be measured in vivo or in vitro assays known to those of ordinary skill in the art. For example, the GLP-1 agonist may be administered to an animal with increased blood glucose (e.g. obtained using an Intravenous Glucose Tolerance Test (IVGTT). A person skilled in the art will be able to determine a suitable glucose dosage and a suitable blood sampling regime, e.g. depending on the species of the animal, for the IVGTT) and measure the plasma insulin concentration overtime. Suitable assays have been described in such as WO2015/155151.

The term half maximal effective concentration (EC₅₀) generally refers to the concentration which induces a response halfway between the baseline and maximum, by reference to the dose response curve. EC₅₀ is used as a measure of the potency of a compound and represents the concentration where 50% of its maximal effect is observed. Due to the albumin binding effects of GLP-1 agonists comprising a substituent as described herein, it is important to pay attention to if the assay includes human serum albumin or not.

The in vitro potency of the GLP-1 agonist may be determined as described in WO2015/155151, Example 29 without Human Serum Albumin (HSA), and the EC₅₀ determined. The lower the EC₅₀ value, the better the potency. In some embodiments the potency (EC50) as determined (without HSA) is 5-1000 pM, such as 10-750 pM, 10-500 pM or 10-200 pM. In some embodiments the EC50 (without HSA) is at most 500 pM, such as at most 300 pM, such as at most 200 pM. In some embodiments the EC50 (without HSA) is comparable to human GLP-1(7-37). In some embodiments the EC50 (without HSA) is at most 50 pM. In a further such embodiment the EC50 is at most 40 pM, such as at most 30 pM such as at most 20 pM, such as at most 10 pM. In some embodiments the EC50 is about 10 pM.

In some embodiments the GLP-1 agonist comprises one or more substitutions, deletions, additions and/or modifications. In some embodiments a modification is a covalently attached substituent. In some embodiments the GLP-1 agonist comprises a peptide which is the human GLP-1 (GLP-1(7-37)) or a variant thereof. Human GLP-1, also referred to herein as “GLP-1(7-37)”, has the sequence HAEGTFTSDV SSYLEGQAAKEFIAWLVKGRG (SEQ ID No: 1). In some embodiments the term “variant” refers to a compound which comprises one or more amino acid substitutions, deletions, additions and/or modifications. Such addition or deletion of amino acid residues may take place at the N-terminal of the peptide and/or at the C-terminal of the peptide. In some embodiments a simple nomenclature is used to describe the GLP-1 agonist, e.g., [Aib8] GLP-1(7-37) designates a GLP-1(7-37) receptor agonist wherein the naturally occurring Ala in position 8 has been substituted with Aib. In some embodiments the GLP-1 agonist comprises a maximum of 12 amino acids which have been altered, e.g., by substitution, deletion, insertion and/or modification, compared to GLP-1(7-37) (SEQ ID No: 1). In some embodiments the GLP-1 agonist comprises a maximum of 10 amino acids which have been altered, e.g., by substitution, deletion, insertion and/or modification, compared to GLP-1(7-37) (SEQ ID No: 1). In some embodiments the GLP-1 agonist comprises a maximum of 9 amino acids which have been altered, e.g., by substitution, deletion, insertion and/or modification, compared to GLP-1(7-37) (SEQ ID No: 1). In some embodiments the GLP-1 agonist comprises a maximum of 8 amino acids which have been altered, e.g., by substitution, deletion, insertion and/or modification, compared to GLP-1(7-37) (SEQ ID No: 1). In some embodiments the GLP-1 agonist comprises a maximum of 7 amino acids which have been altered, e.g., by substitution, deletion, insertion and/or modification, compared to GLP-1(7-37) (SEQ ID No: 1). In some embodiments the GLP-1 agonist comprises a maximum of 6 amino acids which have been altered, e.g., by substitution, deletion, insertion and/or modification, compared to GLP-1(7-37) (SEQ ID No: 1). In some embodiments the GLP-1 agonist comprises a maximum of 5 amino acids which have been altered, e.g., by substitution, deletion, insertion and/or modification, compared to GLP-1(7-37) (SEQ ID No: 1). In some embodiments the GLP-1 agonist comprises a maximum of 4 amino acids which have been altered, e.g., by substitution, deletion, insertion and/or modification, compared to GLP-1(7-37) (SEQ ID No: 1). In some embodiments the GLP-1 agonist comprises a maximum of 3 amino acids which have been altered, e.g., by substitution, deletion, insertion and/or modification, compared to GLP-1(7-37) (SEQ ID No: 1). In some embodiments the GLP-1 agonist comprises a maximum of 2 amino acids which have been altered, e.g., by substitution, deletion, insertion and/or modification, compared to GLP-1(7-37) (SEQ ID No: 1). Unless otherwise stated the GLP-1 comprises only L-amino acids.

In some embodiments the GLP-1 agonist exhibits at least 60%, 65%, 70%, 80% or 90% sequence identity to GLP-1(7-37) over the entire length of GLP-1(7-37). As an example of a method for determination of sequence identity between two compounds, the two peptides [Aib8]GLP-1(7-37) and GLP-1(7-37) are aligned. The sequence identity of [Aib8]GLP-1(7-37) relative to GLP-1(7-37) is given by the number of aligned identical residues minus the number of different residues divided by the total number of residues in GLP-1(7-37). Accordingly, in said example the sequence identity is (31-1)/31.

In some embodiments the C-terminal of the GLP-1 agonist is an amide. In some embodiments the GLP-1 agonist is GLP-1(7-37) or GLP-1(7-36)amide.

In order to prolong the effect of the GLP-1 agonist it is preferred that the GLP-1 agonist have an extended half-life. The half-life can be determined by method known in the art an in an appropriate model, such as in Male Sprague Dawley rats or minipigs as described in WO2012/140117. Half-life in rats may be determined as in Example 39 and the half-life in minipigs may be determined as in Example 37 therein.

In some embodiments the GLP-1 agonist according to the invention has a half-life above 2 hours in rat. In some embodiments the GLP-1 agonist according to the invention has a half-life above 4 hours, such as above 6 hours, such as above 8 hours, such as above 10 hours, such as above 12 hours or such as above 15 hours in rat.

In some embodiments the GLP-1 agonist according to the invention has a half-life above 24 hours in minipig. In some embodiments the GLP-1 agonist according to the invention has a half-life above 30 hours, such as above 36 hours, such as above 42 hours, such as above 48 hours, such as above 54 hours or such as above 60 hours in minipig.

In some embodiments the GLP-1 agonist comprises one or two substituents which are covalently attached to the peptide and wherein said substituent comprises a lipophilic moiety. In some embodiments the substituent comprises a fatty acid or a fatty diacid. In some embodiments the substituent comprises a C16, C18 or C20 fatty acid. In some embodiments the substituent comprises a C16, C18 or C20 fatty diacid.

In some embodiments the substituent comprises a linker (also referred to as a spacer) located proximally in said substituent to the point of attachment between said substituent and the peptide in the GLP-1 agonist. In some embodiments the substituent comprises a linker located proximally in said substituent to the point of attachment between said substituent and the peptide in said GLP-1 agonist. In some embodiments the substituent comprises one or more 8-amino-3,6-dioxaoctanoic acid (OEG), such as two OEG. The one or more OEG may be a linker.

In some embodiments the GLP-1 agonist is selected from the group consisting of semaglutide, Compound A, and Compound B.

In some embodiments the GLP-1 agonist is semaglutide, also known as N-epsilon26-[2-(2-{2-[2-(2-{2-[(S)-4-carboxy-4-(17-carboxyheptadecanoylamino) butyrylamino]ethoxy}ethoxy)acetylamino]ethoxy}ethoxy)acetyl][Aib8,Arg34]GLP-1(7-37), (SEQ ID NO. 4) which may be prepared as described in WO2006/097537, Example 4 with the following structure:

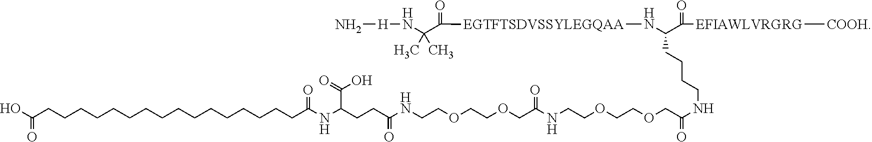

In some embodiments the GLP-1 agonist is Compound A, which is diacylated [Aib8,Arg34,Lys37]GLP-1(7-37) as shown in Example 2 of WO2011/080103 and named N^{ε26}{2-[2-(2-{2-[2-(2-{(S)-4-Carboxy-4-[10-(4-carboxyphenoxy)decanoylamino]butyrylamino}-ethoxy)ethoxy]acetylamino}ethoxy) ethoxy]acetyl}, N^{ϵ37}-{2-[2-(2-{2-[2-(2-{(S)-4-carboxy-4-[10-(4-carboxyphenoxy)decanoylamino] butyrylamino}ethoxy)ethoxy]acetylamino}ethoxy)ethoxy]-acetyl}-[Aib⁸,Arg³⁴,Lys³⁷]GLP-1(7-37)-peptide.

In some embodiments the GLP-1 agonist is Compound B which is Diacylated [Aib8,Glu22,Arg26,Lys27,Glu30,Arg34,Lys36]-GLP-1-(7-37)-peptidyl-Glu-Gly as shown in Example 31 of WO2012/140117 and named N^{ε27}-[2-[2-[2-[[2-[2-[2-[[(4S)-4-carboxy-4-[10-(4-carboxyphenoxy)decanoylamino]butanoyl]amino]ethoxy]ethoxy]acetyl]amino] ethoxy]ethoxy]-acetyl], N^{ε36}-[2-[2-[2-[[2-[2-[2-[[(4S)-4-carboxy-4-[10-(4-carboxyphenoxy) decanoylamino]-butanoyl]amino]ethoxy]ethoxy]acetyl]amino]ethoxy]ethoxy]acetyl]-[Aib8,Glu22,Arg26,Lys27, Glu30,Arg34,Lys36]-GLP-1-(7-37)-peptidyl-Glu-Gly.

In some embodiments the GLP-1 agonist is in the form of any pharmaceutically acceptable salt, amide, or ester thereof. In some embodiments the composition comprises the GLP-1 agonist or a pharmaceutically acceptable salt, amide, or ester thereof. In some embodiments the composition comprises the GLP-1 agonist and one or more pharmaceutically acceptable counter ions.

### SGLT2 Inhibitor

In some embodiments the compositions of the invention comprise an SGLT2 inhibitor. In some embodiments the SGLT2 inhibitor is also an SGLT1 inhibitor. SGLT2 and SGLT1 inhibitors are compounds with the capacity to inhibit the sodium glucose linked transporter. In some embodiments the SGLT2 inhibitor is a glucopyranosyl-substituted benzene derivative. In some embodiments the SGLT2 inhibitor is selected from the group consisting of dapagliflozin, empagliflozin, canagliflozin, ertugliflozin, sotagliflozin, ipragliflozin, tofogliflozin, luseogliflozin, bexagliflozin, and remogloflozin. In some embodiments the SGLT2 inhibitor is dapagliflozin. In some embodiments the SGLT2 inhibitor is empagliflozin. In some embodiments the SGLT2 inhibitor is canagliflozin. In some embodiments the SGLT2 inhibitor is ertugliflozin. In some embodiments the SGLT2 inhibitor is sotagliflozin. In some embodiments the SGLT2 inhibitor is ipragliflozin. In some embodiments the SGLT2 inhibitor is tofogliflozin. In some embodiments the SGLT2 inhibitor is luseogliflozin. In some embodiments the SGLT2 inhibitor is bexagliflozin. In some embodiments the SGLT2 inhibitor is remogloflozin. SGLT2 inhibitors may be prepared according to methods known in the art, for example as shown in WO 2006/120208 WO 2007/031548, or WO2008/002824.

The term “SGLT2 inhibitor” as used herein relates to a compound which provides an inhibitory effect on the sodium-glucose transporter 2 (SGLT2), such as the human SGLT2. In some embodiments inhibitory effect on the human SGLT2, measured as IC50, of the SGLT2 inhibitor is below 1000 nM, such as below 100 nM or below 50 nM. In some embodiments IC50 values of the SGLT2 inhibitor is at least 0.01 nM, such as at least 0.1 nM. Methods for determining inhibitory effect on human SGLT2 are known in the art, e.g. page 23-24 of WO2007/093610. In some embodiments the SGLT2 inhibitor is in an amorphous form. In some embodiments the SGLT2 inhibitor is in the form of a pharmaceutically acceptable salt, hydrate and/or solvate thereof. In some embodiments the SGLT2 inhibitor is in the form of a pharmaceutically acceptable salt, anhydrate and/or solvate thereof. In some embodiments the SGLT2 inhibitor is in a crystalline form, for example of its pharmaceutically acceptable salt, hydrate and/or solvate. In some embodiments the SGLT2 inhibitor or a pharmaceutically acceptable salt thereof is in continuous and/or discontinuous amorphous form or in the form of a solvate or a co-crystal. In some embodiments the term “co-crystal”, as used herein, is a crystalline single-phase material composed of two or more different molecular or ionic compounds. In some embodiments the SGLT2 inhibitor or a pharmaceutically acceptable salt thereof is in continuous and/or discontinuous amorphous form or in the form of a solvate. In some embodiments the SGLT2 inhibitor or a pharmaceutically acceptable salt thereof is in continuous and/or discontinuous amorphous form or in the form of a co-crystal. In some embodiments the composition comprises SGLT2 inhibitor in an amount of 0.5-300 mg, such as 5-200 mg or 10-100 mg.

### Dapagliflozin

In some embodiments the compositions of the invention comprise the SGLT2 inhibitor dapagliflozin. In some embodiments the structure of dapagliflozin is as shown in formula (XII):

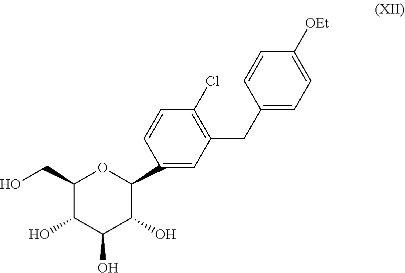 [SMILES: CCOc1ccc(Cc2cc([C@@H]3O[C@H](CO)[C@@H](O)[C@H](O)[C@H]3O)ccc2Cl)cc1]

OH (XII) or a stereoisomer thereof. In some embodiments dapagliflozin is in the form of a pharmaceutically acceptable salt, an ester or a solvate thereof. In some embodiments dapagliflozin or a pharmaceutically acceptable salt thereof is in continuous and/or discontinuous amorphous form or in the form of a solvate or co-crystal. In some embodiments dapagliflozin or a pharmaceutically acceptable salt thereof is in continuous and/or discontinuous amorphous form or in the form of a solvate. In some embodiments dapagliflozin or a pharmaceutically acceptable salt thereof is in continuous and/or discontinuous amorphous form or in the form of a co-crystal. The ester may be a prodrug ester of dapagliflozin, such as an in vivo cleavable ester. A pharmaceutically-acceptable ester may be cleaved in the human or animal body to produce the parent acid (e.g. where said ester is methoxymethyl) or hydroxy group (e.g. where said ester is an acetyl ester). The solvate may comprise or consist of a propylene glycol solvate of dapagliflozin, such as dapagliflozin propylene glycol (1:1). In some embodiments dapagliflozin is in the form of its propylene glycol solvate hydrate (1:1:1). In some embodiments propylene glycol is in the (S) form, the (R) form, or a mixture thereof. In some embodiments propylene glycol is in the (S) form. In some embodiments the composition comprises dapagliflozin and an amino acid selected from the group consisting of glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine; dapagliflozin and the amino acid may be in the form of a co-crystal. In some embodiments the composition comprises dapagliflozin and an amino acid selected from the group consisting of glycine, alanine, valine, leucine, isoleucine, proline, phenylalanine, methionine, and tryptophan. The amino acid may be form of its L- or D-stereoisomer. In some embodiments the composition comprises dapagliflozin and L-proline. In some embodiments the composition comprises dapagliflozin and D-proline. In some embodiments the composition comprises dapagliflozin and a 1,2-alkanediol or a hydrate thereof. In some embodiments the composition comprises dapagliflozin and a compound selected from the group consisting of 1,2-butanediol, 1,2-pentanediol, 1,2-hexanediol, and 1,2-heptanediol or a hydrate or mixture thereof. In some embodiments dapagliflozin is in the form of a solvate comprising 1,2 butanediol or a hydrate thereof. In some embodiments the composition comprises dapagliflozin and a compound selected from the group consisting of (S)-1,2-butanediol, (R)-1,2-butanediol, (S)-1,2-pentanediol, (R)-1,2-pentanediol, (S)-1,2-hexanediol, (R)-1,2-hexanediol, (S)-1,2-heptanediol, (R)-1,2-heptanediol or a hydrate or mixture thereof. In some embodiments the composition comprises dapagliflozin and a compound selected from the group consisting of (S)-1,2-butanediol and (R)-1,2-butanediol or a hydrate or mixture thereof. In some embodiments the composition comprises dapagliflozin and citrate. In some embodiments dapagliflozin is in the form of a co-crystal comprising citrate. In some embodiments compounds referred to herein is an anhydrate unless otherwise mentioned. In some embodiments the composition comprises dapagliflozin in an amount of 0.5-200 mg, such as 5-50 mg. In some embodiments the composition comprises dapagliflozin in an amount of 5 mg or 10 mg. In some embodiments dapagliflozin is administered at a dose from 0.5 to 200 mg/day, such as 3-20 mg/day, 5 mg/day or 10 mg/day.

### Empagliflozin

In some embodiments the compositions of the invention comprise the SGLT2 inhibitor empagliflozin. In some embodiments the structure of empagliflozin is as shown in formula (XIII):

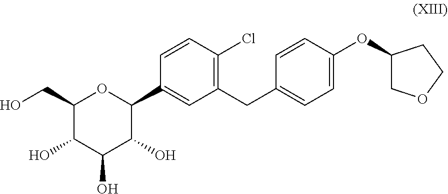 [SMILES: OC[C@H]1O[C@@H](c2ccc(Cl)c(Cc3ccc(O[C@H]4CCOC4)cc3)c2)[C@H](O)[C@@H](O)[C@@H]1O]

or a stereoisomer thereof. In some embodiments empagliflozin is in the form of a pharmaceutically acceptable salt, an ester or a solvate thereof. In some embodiments empagliflozin or a pharmaceutically acceptable salt thereof is in continuous and/or discontinuous amorphous form or in the form of a solvate or co-crystal. In some embodiments empagliflozin or a pharmaceutically acceptable salt thereof is in continuous and/or discontinuous amorphous form or in the form of a solvate. In some embodiments empagliflozin or a pharmaceutically acceptable salt thereof is in continuous and/or discontinuous amorphous form or in the form of a co-crystal. The ester may be a prodrug ester of empagliflozin, such as an in vivo cleavable ester. A pharmaceutically-acceptable ester may be cleaved in the human or animal body to produce the parent acid (e.g. where said ester is methoxymethyl) or hydroxy group (e.g. where said ester is an acetyl ester). In some embodiments the composition comprises empagliflozin in an amount of 0.5-200 mg, such as 5-50 mg. In some embodiments the composition comprises empagliflozin in an amount of 10 mg or 25 mg. In some embodiments empagliflozin is administered at a dose of 0.5-200 mg/day, such as 5-50 mg/day. In some embodiments empagliflozin is administered at a dose of 10 mg/day or 25 mg/day.

### Delivery Agent

A delivery agent or absorption enhancer is for the present invention an excipient capable of increasing the oral exposure of a therapeutic active ingredient, such as a GLP-1 agonist.

### Salt of N-(8-(2-hydroxybenzoyl)amino)caprylic acid

The delivery agent used in the present invention is a salt of N-(8-(2-hydroxybenzoyl)amino)caprylic acid (also referred to herein as a salt of NAC), which contains the anion N-(8-(2-hydroxybenzoyl)amino)caprylate. The structural formula of N-(8-(2-hydroxybenzoyl)amino)caprylate is shown in formula (I).

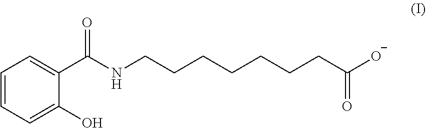 [SMILES: O=C([O-])CCCCCCCNC(=O)c1ccccc1O]

In some embodiments the salt of NAC comprises one monovalent cation, two monovalent cations or one divalent cation. In some embodiments the salt of NAC is selected from the group consisting of the sodium salt, potassium salt and/or ammonium salt of NAC.

In some embodiments the salt of NAC is selected from the group consisting of the sodium salt, potassium salt and/or the ammonium salt. In some embodiments the salt of NAC is the sodium salt or the potassium salt. Salts of N-(8-(2-hydroxybenzoyl)amino)caprylate may be prepared using the method described in e.g. WO96/030036, WO00/046182, WO01/092206 or WO2008/028859.

The salt of NAC may be crystalline and/or amorphous. In some embodiments the delivery agent comprises the anhydrate, monohydrate, dihydrate, trihydrate, a solvate or one third of a hydrate of the salt of NAC as well as combinations thereof. In some embodiments the delivery agent is a salt of NAC as described in WO2007/121318.

In some embodiments the delivery agent is sodium N-(8-(2-hydroxybenzoyl)amino)caprylate (referred to as “SNAC” herein), also known as sodium 8-(salicyloylamino)octanoate.

### Composition

The composition or pharmaceutical composition of the present invention is a solid or dry composition suited for administration by the oral route as described further herein below.

In some embodiments the composition comprises at least one pharmaceutically acceptable excipient. The term “excipient” as used herein broadly refers to any component other than the active therapeutic ingredient(s) or active pharmaceutical ingredient(s) (API(s)). The excipient may be a pharmaceutically inert substance, an inactive substance, and/or a therapeutically or medicinally non-active substance.

The excipient may serve various purposes, e.g. as a carrier, vehicle, filler, binder, lubricant, glidant, disintegrant, flow control agent, crystallization inhibitors solubilizer, stabilizer, colouring agent, flavouring agent, surfactant, emulsifier, delivery agent, hydrotrope or combinations of thereof and/or to improve administration, and/or absorption of the therapeutically active substance(s) or active pharmaceutical ingredient(s). The amount of each excipient used may vary within ranges conventional in the art. Techniques and excipients which may be used to formulate oral dosage forms are described in Handbook of Pharmaceutical Excipients, 8th edition, Sheskey et al., Eds., American Pharmaceuticals Association and the Pharmaceutical Press, publications department of the Royal Pharmaceutical Society of Great Britain (2017); and Remington: the Science and Practice of Pharmacy, 22nd edition, Remington and Allen, Eds., Pharmaceutical Press (2013).

In some embodiments the excipients may be selected from binders, such as polyvinyl pyrrolidone (povidone), etc.; fillers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, etc.; lubricants and/or glidants such as stearic acid, magnesium stearate, sodium stearylfumarate, glycerol tribehenate, etc.; flow control agents such as colloidal silica, talc, etc.; crystallization inhibitors such as Povidone, etc.; solubilizers such as Pluronic, Povidone, etc.; colouring agents, including dyes and pigments such as iron oxide red or yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as Pluronic, polyethylene glycols, sodium carboxymethyl cellulose, polyethoxylated and hydrogenated castor oil, etc.; and mixtures of two or more of these excipients and/or adjuvants.

The composition may comprise a binder, such as povidone; starches; celluloses and derivatives thereof, such as microcrystalline cellulose, e.g., Avicel PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be selected from the group consisting of dry binders and/or wet granulation binders. Suitable dry binders are, e.g., cellulose powder and microcrystalline cellulose, such as Avicel PH 102 and Avicel PH 200. In some embodiments the composition comprises Avicel, such as Aavicel PH 102. Suitable binders for wet granulation or dry granulation are corn starch, polyvinyl pyrrolidone (povidon), vinylpyrrolidone-vinylacetate copolymer (copovidone) and cellulose derivatives like hydroxymethylcellulose, hydroxyethylcellulose, hydroxypropylcellulose and hydroxyl-propylmethylcellulose. In some embodiments the composition comprises povidone.

In some embodiments the composition comprises a filler which may be selected from lactose, mannitol, erythritol, sucrose, sorbitol, calcium phosphate, such as calciumhydrogen phosphate, microcrystalline cellulose, powdered cellulose, confectioner's sugar, compressible sugar, dextrates, dextrin and dextrose. In some embodiments the composition comprises microcrystalline cellulose, such as Avicel PH 102 or Avicel PH 200.

In some embodiments the composition comprises a lubricant and/or a glidant. In some embodiments the composition comprises a lubricant and/or a glidant, such as talc, magnesium stearate, calcium stearate, zinc stearate, glyceryl behenate, glyceryl debehenate, behenoyl polyoxyl-8 glycerides, polyethylene oxide polymers, sodium lauryl sulfate, magnesium lauryl sulfate, sodium oleate, sodium stearyl fumarate, stearic acid, hydrogenated vegetable oils, silicon dioxide and/or polyethylene glycol etc. In some embodiments the composition comprises magnesium stearate or glyceryl debehenate (such as the product Compritol® 888 ATO).

In some embodiments the composition comprises a disintegrant, such as sodium starch glycolate, polacrilin potassium, sodium starch glycolate, crospovidon, croscarmellose, sodium carboxymethylcellulose or dried corn starch. The composition may comprise one or more surfactants, for example a surfactant, at least one surfactant, or two different surfactants. The term “surfactant” refers to any molecules or ions that are comprised of a water-soluble (hydrophilic) part, and a fat-soluble (lipophilic) part. The surfactant may e.g. be selected from the group consisting of anionic surfactants, cationic surfactants, nonionic surfactants, and/or zwitterionic surfactants.

### Hydrotropes

The composition of the present invention comprises one or more hydrotropes. Hydrotrope, like surfactants, comprise both a hydrophilic part and a hydrophobic and can form micelles and self-aggregate, however they solubilize solutes without micellar solubilization. The inventors have found that the absorption of the GLP-1 agonist and thus the plasma exposure can be increased by including a hydrotrope in the compositions. Without being bound by theory, it is contemplated that the hydrotrope increases the solubility of the delivery agent, such as a salt of NAC, as exemplified by SNAC herein. As shown by Assay I herein, hydrotropes can increase the solubility of SNAC in water.

In some embodiments the hydrotrope is capable of increasing the solubility of SNAC. In some embodiments the hydrotrope is capable of increasing the solubility of a salt of NAC, such as SNAC, at least 2-fold at a concentration of 200 mg/ml at pH 6 at room temperature. In some embodiments, the hydrotrope increases solubility of a salt of NAC, such as SNAC, at least 3-, 4- or 5-fold when measured as described in Assay I herein. In some embodiments, the hydrotrope increase the solubility of SNAC at least 5-fold, such as 8-fold or such as 10-fold when measured as described in Assay I.

In some embodiments the molecular weight of the hydrotrope is at most 400 g/mol or such as at most 250 g/mol.

In some embodiments the molecular weight of the hydrotrope is at least 80 g/mol or such as at least 100 g/mol

In some embodiments the hydrotrope comprises an aromatic ring structure.

In some embodiments the hydrotrope has a similar molecular structure as nicotinamide and Resorcinol, which both comprises an aromatic ring structure.

Included herein are also a physiologically acceptable salt thereof, such as the sodium, potassium, chloride or sulphate salt.

In some embodiments the one or more hydrotrope has the structure of Chem I

### Chem I:

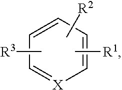

wherein

### X is CH or N,

R¹, R² and R³ are independently selected from: —H, —OH, —CO₂H, —CON(R⁴)₂, —SO₃H and —CH₃, wherein R⁴ is —H, —CH₃ or —CH₂—CH₃
or a physiologically acceptable salt thereof.

In some embodiments, where the structure is Chem I,

### X is CH or N,

R¹ is selected from —OH, —SO₃H and CON(R⁴)₂, wherein R⁴ is —H, —CH₃ or —CH₂—CH₃,
R2 is selected from: —OH and —H and
R3 is selected from: —H, —OH and —CH₃ or a physiologically acceptable salt thereof.

In some embodiments, the hydrotrope has the structure of Chem I, wherein

### X is CH,

R1 is selected from: —OH and —SO₃H,
R2 and R3 are independently selected from: —H, —OH and —CH₃ or a physiologically acceptable salt thereof.

In some embodiments the one or more hydrotrope has the structure of Chem II

### Chem II:

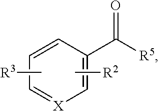

wherein

### X is CH or N

R² and R³ are independently selected from: —H, —OH and —CH₃
R⁵ is selected from: —OH and N(R⁴)₂, wherein R4 is —H, —CH₃ or —CH₂—CH₃ or a physiologically acceptable salt thereof.

In some embodiments, the one or more hydrotrope has the structure of Chem II wherein,

### X is CH

### R5 is —OH and

R2 and R3 are independently selected from: —OH and —H or a physiologically acceptable salt thereof.

In some embodiments, the one or more hydrotrope has the structure of Chem II as defined above, with the proviso that the hydrotrope is not sodium benzoate.

In some embodiments, the one or more hydrotrope has the structure of Chem II, wherein

### X is N,

R⁵ is selected from: —OH and N(R⁴)₂, wherein R4 is —H, —CH₃ or —CH₂—CH₃
R² and R³ are independently selected from: —H, —OH and —CH₃ or
a physiologically acceptable salt thereof.

In some embodiments, the one or more hydrotrope has the structure of Chem II, wherein

### X is N,

R⁵ is NH₂, and
R² and R³ are independently selected from: —H, —OH and —CH₃ or
a physiologically acceptable salt thereof.

In some embodiments the one or more hydrotrope has the structure of Chem III

### Chem III:

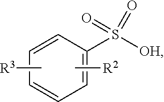

wherein
R² and R³ are independently selected from —H and —CH₃.

In some embodiments the one or more hydrotrope has the structure of Chem IV

### Chem IV:

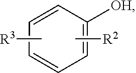

wherein
R2 and R3 are independently selected from: —H and —OH.

In some embodiments the hydrotrope or hydrotropes are selected from the group consisting of: Resorcinol, Pyrocathecol, N,N dimethyl benzamide, Pyrogallol, Nicotinamide. Tannic acid, Epigallocatechin gallate, N,N diethyl nicotinamide, Gentisic acid sodium salt hydrate, N,N dimethyl urea, 1-Methylnicotinamide, Sodium Xylenesulfonate, and Sodium p-toluenesulfonate, Sodium salicylate, Nipecotamide, p-hydroxybenzoic acid sodium, and Sodium benzoate. In some embodiments the hydrotrope or hydrotropes are selected from the group consisting of: Resorcinol, Pyrocathecol, N,N dimethyl benzamide, Pyrogallol, Nicotinamide, Tannic acid, Epigallocatechin gallate, N,N diethyl nicotinamide, Gentisic acid sodium salt hydrate, N,N dimethyl urea, 1-Methylnicotinamide, Sodium Xylenesulfonate, and Sodium p-toluenesulfonate. In some embodiments the hydrotrope or hydrotropes are selected from the group consisting of: Resorcinol, Pyrocathecol, N,N dimethyl benzamide, Pyrogallol, Nicotinamide, Tannic acid, Epigallocatechin gallate, and N,N diethyl nicotinamide. In some embodiments the hydrotrope or hydrotropes are selected from the group consisting of: Resorcinol, Pyrocathecol, N,N dimethyl benzamide, Pyrogallol, and Nicotinamide.

In some embodiments the hydrotrope or hydrotropes are selected from the group consisting of: Nipecotamide, Nicotinamide, p-hydroxybenzoic acid sodium, N,N dimethyl urea, N,N dimethyl benzamide, N,N diethyl nicotinamide, Sodium salicylate, Resorcinol, Sodium benzoate, Sodium Xylenesulfonate, Sodium p-toluenesulfonate, 1-Methylnicotinamide, Pyrogallol, Pyrocathecol, Epigallocatechin gallate, Tannic acid and Gentisic acid sodium salt hydrate. In some embodiments the hydrotrope or hydrotropes are selected from the group consisting of: Nipecotamide, Nicotinamide, p-hydroxybenzoic acid sodium, N,N dimethyl urea, N,N dimethyl benzamide, N,N diethyl nicotinamide, Sodium salicylate, Resorcinol, Sodium benzoate, Sodium Xylenesulfonate, Sodium p-toluenesulfonate, 1-Methylnicotinamide, Pyrogallol, Pyrocathecol, Epigallocatechin gallate, Tannic acid and Gentisic acid sodium salt hydrate. In some embodiments the hydrotrope or hydrotropes are selected from the group consisting of: Nicotinamide, p-hydroxybenzoic acid sodium, N,N dimethyl urea, N,N dimethyl benzamide, N,N diethyl nicotinamide, Sodium salicylate, Resorcinol, Sodium benzoate, Sodium Xylenesulfonate, Sodium p-toluenesulfonate, 1-Methylnicotinamide, Pyrogallol, Pyrocathecol, Epigallocatechin gallate and Gentisic acid sodium salt hydrate. In some embodiments the hydrotrope or hydrotropes are selected from the group consisting of: Nicotinamide, N,N dimethyl benzamide, N,N diethyl nicotinamide, Resorcinol, Sodium benzoate, Sodium Xylenesulfonate, Sodium p-toluenesulfonate, 1-Methylnicotinamide, Pyrogallol, Pyrocathecol and Gentisic acid sodium salt hydrate. In some embodiments the hydrotrope or hydrotropes is/are selected from the group consisting of: Resorcinol, Pyrocatechol, Pyrogallol, Gentisic acid, Xylenesulfonate, p-toluenesulfonate, Nicotinamide, Dimethylbenzamide, Diethylbenzamide, 1-methylnicotinamide, Salicyclic acid, P-Hydroxybenzoic acid and Benzoate. In some embodiments the hydrotrope or hydrotropes is/are selected from the group consisting of: Resorcinol, Pyrocatechol, Pyrogallol, Gentisic acid, Xylenesulfonate, p-toluenesulfonate, Nicotinamide, Dimethylbenzamide, Diethylbenzamide, 1-methylnicotinamide, Salicyclic acid and P-Hydroxybenzoic acid.

In some embodiments the hydrotrope or hydrotropes is/are selected from the group consisting of: Resorcinol, Pyrocatechol and Pyrogallol. In some embodiments the hydrotrope or hydrotropes is/are selected from the group consisting of: Xylenesulfonate and p-toluenesulfonate. In some embodiments the hydrotrope or hydrotropes is/are selected from the group consisting of: Nicotinamide, Dimethylbenzamide, Diethylbenzamide and 1-methylnicotinamide. In some embodiments the hydrotrope or hydrotropes is/are selected from the group consisting of: Gentisic acid, Salicyclic acid, P-Hydroxybenzoic acid and Benzoate. In some embodiments the hydrotrope or hydrotropes is/are selected from the group consisting of: Gentisic acid, Salicyclic acid and P-Hydroxybenzoic acid. In some embodiments the hydrotrope or hydrotropes are nicotinamide and/or Resorcinol. In some embodiments the hydrotrope is nicotinamide. In some embodiments the hydrotrope is not sodium benzoate.

As shown in the examples herein, the composition of the invention comprises a GLP-1 agonist, a SGLT2 inhibitor, a delivery agent and a hydrotrope.

The description here below also refers to compositions consisting of specific ingredients, the GLP-1 agonist, the SGLT2 inhibitor, the delivery agent and the hydrotrope and optionally a lubricant, the term consisting is to be understood to nevertheless encompass trace amounts of any substance with no effect on the function of the composition. Such substances can be impurities remaining in preparation of the GLP-1 agonist, the SGLT2 inhibitor, from the production of the salt of NAC, the hydrotrope preparation or minimal amounts of any pharmaceutical acceptable excipient that do not affect the quality or absorption of the formulation.

In some embodiments the pharmaceutical composition comprises a balanced amount of the hydrotrope relative to the amount of the delivery agent. The effect of the hydrotrope has been observed over a range of concentrations.

In some embodiments the ratio of salt of NAC/hydrotrope (w/w) is at least 0.5, such as at least 0.75 or such as at least 1. In some embodiments the ratio of salt of NAC/hydrotrope (w/w) is 0.5-10.0 or such as such as 0.5-8 or such as 0.5-5. In some embodiments the ratio of salt of NAC/hydrotrope (w/w) is 0.5-10.0 or such as 0.75-10.0, 0.5-8.0 or 1-2.0. In some embodiments the ratio of salt of NAC/hydrotrope (w/w) is 1.3-1.7.

In some embodiments the ratio of SNAC/Nicotinamide (w/w) is at least 0.5, such as at least 0.75, such as at least 1. In some embodiments the ratio of salt of SNAC/Nicotinamide (w/w) is 0.5-10.0 or such as 0.5-8 or such as 0.5-5. In some embodiments the ratio of salt of SNAC/Nicotinamide (w/w) is 0.5-10.0 or such as 0.75-10.0, 0.5-8.0 or 1-2.0.

In some embodiments the ratio of SNAC/Resorcinol (w/w) is at least 0.5, such as at least 0.75, such as at least 1. In some embodiments the ratio of salt of SNAC/Resorcinol (w/w) is 0.5-10.0 or such as 0.75-10.0, 0.5-8.0 or such as 1-2.0.

In some embodiments the ratio of hydrotrope/salt of NAC (w/w) is at least 0.1, such as at least 0.2 or such as at least 0.3. In some embodiments the ratio of hydrotrope/salt of NAC (w/w) is 0.1-5.0 or such as 0.1-4.0, 0.2-3.0 or 0.25-2.0.

In some embodiments the ratio of Nicotinamide/SNAC (w/w) is at least 0.1-5.0 or such as 0.1-4.0, 0.2-3.0 or 0.25-2.0. In some embodiments the ratio of Nicotinamide/SNAC (w/w) is 0.1-5.0 or such as 0.1-4.0, 0.2-3.0 or 0.25-2.0.

In some embodiments the ratio of Resorcinol/SNAC (w/w) is at least 0.1-5.0 or such as 0.1-4.0, 0.2-3.0 or 0.25-2.0. In some embodiments the ratio of Resorcinol/SNAC (w/w) is 0.1-5.0 or such as 0.1-4.0, 0.2-3.0 or 0.25-2.0.

The amount of lubricant maybe be given relative to the amount of other excipients, e.g. hydrotrope and delivery agent, and not including the active pharmaceutical ingredients (i.e. GLP-1 agonist and SGLT2 inhibitor). Relatively small amounts of the lubricant are usually included, such as less than 5% of the total weight of other excipients.

In some embodiments the composition comprises less than 5 w/w % lubricant of the total amount of delivery agent and hydrotrope. In some embodiments the composition comprises 0.15-5 w/w %, such as 0.25-4 w/w %, lubricant of the total amount of delivery agent and hydrotrope. In some embodiments the composition comprises 0.15-5 w/w %, such as 0.25-4 w/w %, lubricant of the amount of salt of NAC (such as SNAC) and nicotinamide or resorcinol.

The pharmaceutical composition according to the invention is preferably produced in a dosage form suitable for oral administration as described herein below. In the following the absolute amounts of the ingredients of the composition of the invention are provided with reference to the content in a dosage unit i.e. per tablet, capsule or sachet.

The pharmaceutical compositions of the invention may in some embodiments comprise at most 1000 mg of said salt of NAC per dose unit. In some embodiments the invention relates to a composition wherein a dose unit comprises at most 600 mg of said salt of NAC.

In some embodiments the amount of the salt of NAC per dose unit is at least 0.15 mmol, such as selected from the group consisting of at least 0.20 mmol, at least 0.25 mmol, at least 0.30 mmol, at least 0.35 mmol, at least 0.40 mmol, at least 0.45 mmol, at least 0.50 mmol, at least 0.55 mmol and at least 0.60 mmol.

In some embodiments the amount of the salt of NAC per dosage unit of the composition is up to 2 mmol, such as selected from the group consisting of up to 1.5 mmol, up to 1 mmol, up to 0.75 mmol, up to 0.6 mmol, up to 0.5 mmol, up to 0.4 mmol, up to 0.3 mmol and up to 0.2 mmol.

In some embodiments the amount of the salt of N-(8-(2-hydroxybenzoyl) amino)caprylic acid per dose unit of the composition is in the range of 0.20-0.5 mmol, 0.25-1.0 mmol, 0.30-0.75 mmol, or such as 0.45-0.65 mmol.

In some embodiments the amount of SNAC in the composition is at least 50 mg, such as selected from the group consisting of at least 75 mg, at least 100 mg, at least 125 mg, at least 150 mg, at least 175 mg, at least 200 mg, at least 225 mg, at least 250 mg, at least 275 mg and at least 300 mg per dose unit.

In some embodiments the amount of SNAC in the composition is up to 600 mg, such as selected from the group consisting of up to 550 mg, up to 525 mg, up to 500 mg, up to 475 mg, up to 450 mg, up to 425 mg, up to 400 mg, up to 375 mg, up to 350 mg, up to 325 mg per dose unit, and up to 300 mg per dose unit.

In some embodiments the amount of SNAC in the composition is in the range of 75-600 mg. In some embodiments the amount of SNAC in the composition is in the range of 75-400 mg, such as from 80-350 mg, such as from around 100 to around 300 mg per dose unit.

In some embodiments, a dose unit of the pharmaceutical compositions of the invention comprises 0.1-100 mg of the GLP-1 agonist.

In some embodiments a dose unit of the composition comprises an amount of GLP-1 agonist is in the range of 0.1-50 mg, 0.2 to 50 mg, 0.5 to 50 mg or 1 to 40 mg.

In some embodiments a dose unit of the composition comprises an amount of GLP-1 agonist is in the range of 0.1-50 mg, 0.1-40 mg, 0.1-30 mg or 0.1-20 mg.

In some embodiments a dose unit comprises 0.5-5 mg of the GLP-1 agonist, such as 0.75-4.5 mg, such as 1, 1.5, 2, 2.5 or 3 mg or 3.5, 4, 4.5 mg, such as 1-3 or 3-5 mg of the GLP-1 agonist per dose unit. In some embodiments a dose unit comprises 1-20 mg of the GLP-1 agonist, such as 2-15 mg or 3-12 mg of the GLP-1 agonist per dose unit. In some embodiments a dose unit comprises 1-10 mg of the GLP-1 agonist, such as 2-8 mg or 3-6 mg of the GLP-1 agonist per dose unit.

In some embodiments a dose unit comprises 2 to 20 mg of the GLP-1 agonist, such as 2-15 mg, such as 2, 3, 4 or 5 mg, or such as 8, 10, 12 or 14 mg, such as 15 mg or such as 20 mg of the GLP-1 agonist per dose unit.

In some embodiments a dose unit comprises 5 to 50 mg of the GLP-1 agonist, such as 10-45 mg, such as 20, 30 or 40 mg, or such as 25, 35, or 45 mg, or such as 30-50 mg or such as 20-40 mg of the GLP-1 agonist per dose unit.

In some embodiments a dose unit comprises 1-50 mg, such as 3-30 mg or 5-25 mg SGLT2 inhibitor per dose unit. In some embodiments a dose unit comprises 5 or 10 mg of the SGLT2 inhibitor dapagliflozin per dose unit. In some embodiments a dose unit comprises 10 or 25 mg of the SGLT2 inhibitor empagliflozin per dose unit.

As described above the amount of the hydrotrope is to be balanced with the amount of the delivering agents, such as SNAC, but in general a dose unit of the compositions of the invention comprises 10-600 mg of the hydrotrope. In some embodiments a dose unit comprises 20-400 mg, such as 40-300 mg or 50-200 mg, of the hydrotrope. In some embodiments a dose unit comprises 100-600 mg, such as 100-500, such as 150-400 mg, of the hydrotrope. In some embodiments a dose unit comprises 150-300 mg, such as 150-250 mg or about 200 mg, of the hydrotrope. In some embodiments a dose unit comprises 20-150 mg, such as 40-100 mg or 50-90 mg, of the hydrotrope.

In some embodiments, a unit dose of the composition according to the invention comprises 50-600 mg nicotinamide and/or resorcinol. In some embodiments a dose unit comprises 50-400 mg, such as 100-300 mg or 150-250 mg, nicotinamide and/or resorcinol. In some embodiments a dose unit comprises 50-400 mg, such as 50-300, such as 50-200 mg, such as 50-175 mg nicotinamide and/or resorcinol.

In some embodiments a unit dose of the composition according to the invention comprises 50-600 mg nicotinamide. In some embodiments a dose unit comprises 50-400 mg, such as 100-300 mg or 150-250 mg, nicotinamide. In some embodiments a dose unit comprises 50-400 mg, such as 50-300, such as 50-200 mg, such as 50-175 mg nicotinamide.

In some embodiments a unit dose of the composition according to the invention comprises: 0.1-50 mg GLP-1 agonist, 1-50 mg SGLT2 inhibitor, 25-600 mg salt of NAC (such as the sodium salt of NAC (SNAC)), 20-400 mg nicotinamide or resorcinol, and 0-20 mg lubricant.

In some embodiments a unit dose of the composition according to the invention comprises:

i) 0.1-50 mg semaglutide,

ii) 1-50 mg SGLT2 inhibitor,

iii) 25-600 mg salt of NAC (such as the sodium salt of NAC (SNAC)),

iv) 20-400 mg, such as 50-400 mg, nicotinamide or resorcinol, and

v) 0-20 mg lubricant.

In some embodiments a unit dose of the composition according to the invention comprises:

i) 0.1-50 mg semaglutide,

ii) 1-50 mg SGLT2 inhibitor,

iii) 150-600 mg salt of NAC (such as the sodium salt of NAC (SNAC)),

iv) 100-400 mg nicotinamide or resorcinol, and

v) 0-20 mg lubricant.

The amount of GLP-1 agonist may be varied depending on identity of the GLP-1 agonist and the effect desired, i.e. a higher content may be relevant for treating obesity compared to diabetes.

In some embodiments a unit dose of the composition comprises 200-400 mg SNAC, 0.5-50 mg GLP-1 agonist, 1-50 mg SGLT2 inhibitor, 100-400 mg hydrotrope and 1-16 mg lubricant.

In some embodiments a unit dose of the composition comprises 200-400 mg SNAC, 1.5-50 mg GLP-1 agonist, 1-50 mg SGLT2 inhibitor, 100-400 mg hydrotrope and 1-16 mg lubricant.

In some embodiments a unit dose of the composition comprises 200-400 mg SNAC, 5-50 mg GLP-1 agonist, 1-50 mg SGLT2 inhibitor, 100-400 mg hydrotrope and 1-16 mg lubricant.

In some embodiments a unit dose of the composition comprises 200-400 mg SNAC, 0.5-10 mg GLP-1 agonist, 1-50 mg SGLT2 inhibitor, 100-400 mg nicotinamide and 1-16 mg lubricant.

In some embodiments a unit dose of the composition comprises 200-400 mg SNAC, 1.5-50 mg GLP-1 agonist, 1-50 mg SGLT2 inhibitor, 100-400 mg nicotinamide and 1-16 mg lubricant.

In some embodiments a unit dose of the composition comprises 200-400 mg SNAC, 5-50 mg GLP-1 agonist, 1-50 mg SGLT2 inhibitor, 100-400 mg nicotinamide and 1-16 mg lubricant.

In some embodiments a unit dose of the composition according to the invention comprises:

i) 0.1-50 mg semaglutide,

ii) 1-50 mg SGLT2 inhibitor,

iii) 25-400 mg salt of NAC (such as the sodium salt of NAC (SNAC)),

iv) 20-300 mg nicotinamide or resorcinol, and

v) 0-14 mg lubricant.

The amount of GLP-1 agonist may be varied depending on identity of the GLP-1 agonist and the effect desired, i.e. a higher content may be relevant for treating obesity compared to diabetes.

In some embodiments a unit dose of the composition comprises 80-120 mg SNAC, 0.5-5 mg GLP-1 agonist, 1-50 mg SGLT2 inhibitor, 20-200 mg hydrotrope and 0.25-8 mg lubricant.

In some embodiments a unit dose of the composition comprises 80-120 mg SNAC, 1.5-10 mg GLP-1 agonist, 1-50 mg SGLT2 inhibitor, 20-200 mg hydrotrope and 0.25-8 mg lubricant.

In some embodiments a unit dose of the composition comprises 80-120 mg SNAC, 5-50 mg GLP-1 agonist, 1-50 mg SGLT2 inhibitor, 20-200 mg hydrotrope and 0.25-8 mg lubricant.

In some embodiments a unit dose of the composition comprises 80-120 mg SNAC, 0.5-5 mg GLP-1 agonist, 1-50 mg SGLT2 inhibitor, 20-200 mg nicotinamide and 0.25-8 mg lubricant.

In some embodiments a unit dose of the composition comprises 80-120 mg SNAC, 1.5-10 mg GLP-1 agonist, 1-50 mg SGLT2 inhibitor, 20-200 mg nicotinamide and 0.25-8 mg lubricant.

In some embodiments a unit dose of the composition comprises 80-120 mg SNAC, 5-50 mg GLP-1 agonist, 1-50 mg SGLT2 inhibitor, 20-200 mg nicotinamide and 0.25-8 mg lubricant.

### Dosage Form

The composition may be administered in several dosage forms, for example as a tablet; a coated tablet; a sachet or a capsule such as hard or soft gelatine capsule and all such compositions are considered solid oral dosage forms.

The composition may further be compounded in a drug carrier or drug delivery system, e.g. in order to improve stability and/or solubility or further improve exposure. The composition may be a freeze-dried or spray-dried composition.

The composition may be in the form of a dose unit, such as tablet. In some embodiments the weight of the unit dose is in the range of 50 mg to 1000 mg, such as in the range of 50-750 mg, or such as about 100-500 mg. In some embodiments the weight of the dose unit is in the range of 75 mg to 350 mg, such as in the range of 50-300 mg or 100-400 mg. In some embodiments the weight of the dose unit is in the range of 300 mg to 800 mg, such as in the range of 400-700 mg or 500-600 mg.

In some embodiments the composition may be granulated prior to being compressed to tablets. The composition may comprise a granular part and/or an extra-granular part, wherein the granular part has been granulated and the extra-granular part has been added after granulation.

The granular part may comprise the GLP-1 agonist, a SGLT2 inhibitor, the delivery agent and/or the hydrotrope. In some embodiments the granular part may comprise a further excipient, such as a lubricant and/or glidant. In some embodiments the granular part comprises a SGLT2 inhibitor, the delivery agent and the hydrotrope. In some embodiments the granular part comprises the delivery agent and the hydrotrope.

In some embodiments the hydrotrope is included in the granular part, the extra-granular part or both.

In some embodiments the granular part comprises magnesium stearate or glyceryl dibehenate.

The GLP-1 agonist may be included in the granular part or the extra-granular part. In some embodiments the extra-granular part comprises the GLP-1 agonist. The SGLT2 inhibitor may be included in the granular part or the extra-granular part. In some embodiments the extra-granular part comprises the SGLT2 inhibitor. In some embodiments the extra-granular part may further comprise a lubricant and/or a glidant. In some embodiments the granular part may comprise a lubricant and/or a glidant. In some embodiments the granular part and the extra-granular part comprise a lubricant and/or a glidant.

In some embodiments the lubricant and/or a glidant is magnesium stearate or glyceryl dibehenate.

### Preparation of Composition

Preparation of a composition according to the invention may be performed according to methods known in the art.

To prepare a dry blend of tabletting material, the various components are weighed, optionally delumped or sieved and then combined. The mixing of the components may be carried out until a homogeneous blend is obtained.

The term “granules” refers broadly to pharmaceutical ingredients in the form of particles, granules and aggregates which are used in the preparation of solid dose formulations. Generally, granules are obtained by processing a powder or a blend to obtain a solid which is subsequently broken down to obtain granules of desired size.

If granules are to be used in the tabletting material, granules may be produced in a manner known to a person skilled in the art, for example using wet granulation methods known for the production of “built-up” granules or “broken-down” granules. Methods for the formation of built-up granules may operate continuously and comprise, for example simultaneously spraying the granulation mass with granulation solution and drying, for example in a drum granulator, in pan granulators, on disc granulators, in a fluidized bed, by spray-drying or spray-solidifying, or operate discontinuously, for example in a fluidized bed, in a rotary fluid bed, in a batch mixer, such as a high shear mixer or a low shear mixer, or in a spray-drying drum. Methods for the production of broken-down granules, which may be carried out continuously or discontinuously and in which the granulation mass first forms a wet aggregate with the granulation solution, which is subsequently comminuted or by other means formed into granules of the desired size and the granules may then be dried. Suitable equipment for the granulation step are, but not limited to, planetary mixers, low shear mixers, high shear mixers, extruders and spheronizers, such as an apparatus, but not limited to, from the companies Loedige, Glatt, Diosna, Fielder, Collette, Aeschbach, Alexanderwerk, Ytron, Wyss & Probst, Werner & Pfleiderer, HKD, Loser, Fuji, Nica, Caleva and Gabler. Granules may be also formed by dry granulation techniques in which one or more of the excipient(s) and/or the active pharmaceutical ingredient is compressed to form relatively large mouldings, for example slugs or ribbons, which are comminuted by grinding, and the ground material serves as the tabletting material to be later compacted. Suitable equipment for dry granulation is roller compaction equipment from Gerteis, but not limited hereto, such as Gerteis MICRO-PACTOR, MINI-PACTOR and MARCO-PACTOR.

Further methods of obtaining granules can include hot melt extrusion, spray drying, spray granulation and/or ball milling.

In some embodiments the invention relates to a composition comprising

i) semaglutide,

ii) 1-50 mg SGLT2 inhibitor,

iii) a salt of NAC, and

iv) a hydrotrope,

wherein the composition comprises a granulate of iii) and iv), or of ii) and iii) and iv), or of i) and ii) and iii) and iv).

In some embodiments where the granular part comprises both the delivery agent and the hydrotrope, these excipients may be co-processed prior to or in the preparation of the granules. In some embodiments the granular part comprises the delivery agent and the hydrotrope and SGLT2 inhibitor, these ingredients may be co-processed prior to or during the preparation of the granules.

The granulation maybe be obtained by various methods as described above, wherein iii) and iv) are optionally initially mixed either as powders or by the preparation of one ore more solutions comprising optionally both ingredients.

Granules of iii) and iv) or of ii) and iii) and iv) or of i) and ii) and iii) and iv) may then be obtained by dry granulation of the blend, such as by roller compaction. In some embodiments the ingredients may be hot melt extruded to obtain an extrudate which is optionally subsequently milled to obtain the granules. This material can then be used directly or in dry granulation/roller compaction process to obtain the final granules.

In some embodiments a solution of individual ingredients or of iii) and iv) or of ii) and iii) and iv) or of i) and ii) and iii) and iv) is prepared and subject to spray granulation whereby granules are directly obtained. Alternatively, one or more solutions can be used in a fluid bed spray granulation process. In some embodiments spray drying can be used followed by dry granulation/roller compaction to obtain the granules.

In order to obtain a homogenous product one or more sieving step(s) can be included prior to the final dry granulation step/roller compaction or tablet compression.

To compact the tabletting material into a solid oral dosage form, for example a tablet, a tablet press may be used. In a tablet press, the tabletting material is filled (e.g. force fed or gravity fed) into a die cavity. The tabletting material is then compacted by a set of punches applying pressure. Subsequently, the resulting tablet is ejected from the tablet press. The above-mentioned tabletting process is subsequently referred to herein as the “compression process”. Suitable tablet presses include, but are not limited to, rotary tablet presses and eccentric tablet presses. Examples of tablet presses include, but are not limited to, the Fette 102i (Fette GmbH), the Korsch XL100, the Korsch PH 106 rotary tablet press (Korsch AG, Germany), the Korsch EK-O eccentric tabletting press (Korsch AG, Germany) and the Manesty F-Press (Manesty Machines Ltd., United Kingdom).

In some embodiments the invention relates to a method of preparation a composition according to the invention. In some embodiments the method of preparing a tablet comprises; a) granulation of the delivery agent and the hydrotrope and optionally a SGLT2 inhibitor b) blending of the granulates of a) with a GLP-1 agonist and optionally with a SGLT2 inhibitor, and then c) compression of the blend into tablets. The granulation may be a wet or dry granulation. As described above a lubricant such as magnesium stearate or glyceryl behenate may be included in steps a), b) and/or c).

In some embodiments the invention relates to a method for producing a solid pharmaceutical composition comprising the steps of:
i) obtaining a blend comprising a salt of NAC and a hydrotrope, or using separate feeders that assure the right ratio between a salt of NAC and a hydrotrope,
ii) co-processing said salt of NAC and a hydrotrope of i) and
iii) preparing said solid pharmaceutical composition using the product of ii), wherein said solid pharmaceutical composition comprises the GLP-1 agonist semaglutide and an SGLT2 inhibitor.

In some embodiments the method is for producing a solid pharmaceutical composition comprising the steps of:
i) obtaining a blend comprising a salt of NAC and a hydrotrope, or using separate feeders that assure the right ratio between a salt of NAC and a hydrotrope,
ii) hot melt extruding said salt of NAC and a hydrotrope of i) and
iii) preparing said solid pharmaceutical composition, such as tablets, using the extrudate of ii), wherein said solid pharmaceutical composition comprises the GLP-1 agonist semaglutide and an SGLT2 inhibitor.

In some embodiments the composition of the invention is prepared by processing a solution of said salt of NAC and said hydrotrope (such as nicotinamide or resorcinol) by spouted bed or fluid bed spray granulation. In some embodiments the composition of the invention is prepared by preparation of solutions of said salt of NAC and said hydrotrope (such as nicotinamide or resorcinol), said solutions then (a) processed by spouted bed or fluid bed spray granulation and (b) said solutions optionally fed into the process of (a) as separate solutions.

The method may as described herein include further steps, such as a step of admixing the extrudate of ii) with an active pharmaceutical ingredient and optionally any further excipients and preparing said solid pharmaceutical composition using the mixture. The method may as described herein include further steps, such as a step of adding SGLT2 inhibitor when co-processing the salt of NAC and a hydrotrope, followed by admixing the extrudate of ii) with one or more active pharmaceutical ingredients and optionally any further excipients and preparing said solid pharmaceutical composition using the mixture.

### Pharmaceutical Indications

The present invention also relates to a composition of the invention for use as a medicament. In some embodiments the composition of the invention may be used for the following medical treatments, all preferably relating one way or the other to diabetes:

(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;

(ii) delaying or preventing diabetic disease progression, such as progression in type 2 diabetes, delaying the progression of impaired glucose tolerance (IGT) to insulin requiring type 2 diabetes, and/or delaying the progression of non-insulin requiring type 2 diabetes to insulin requiring type 2 diabetes;

(iii) improving β-cell function, such as decreasing β-cell apoptosis, increasing β-cell function and/or β-cell mass, and/or for restoring glucose sensitivity to β-cells;

(iv) prevention and/or treatment of cognitive disorders;

(v) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; and/or delaying gastric emptying;

(vi) prevention and/or treatment of diabetic complications, such as neuropathy, including peripheral neuropathy; nephropathy; or retinopathy;

(vii) improving lipid parameters, such as prevention and/or treatment of dyslipidemia, lowering total serum lipids; lowering HDL; lowering small, dense LDL; lowering VLDL: lowering triglycerides; lowering cholesterol; increasing HDL; lowering plasma levels of lipoprotein a (Lp(a)) in a human; inhibiting generation of apolipoprotein a (apo(a)) in vitro and/or in vivo;

(iix) prevention and/or treatment of cardiovascular diseases, such as syndrome X; atherosclerosis; myocardial infarction; coronary heart disease; stroke, cerebral ischemia; an early cardiac or early cardiovascular disease, such as left ventricular hypertrophy; coronary artery disease; essential hypertension; acute hypertensive emergency; cardiomyopathy; heart insufficiency; exercise tolerance; chronic heart failure; arrhythmia; cardiac dysrhythmia; syncopy; atherosclerosis; mild chronic heart failure; angina pectoris; cardiac bypass reocclusion; intermittent claudication (atherosclerosis obliterans); diastolic dysfunction; and/or systolic dysfunction;

(ix) prevention and/or treatment of gastrointestinal diseases, such as inflammatory bowel syndrome; small bowel syndrome, or Crohn's disease; dyspepsia; and/or gastric ulcers;

(x) prevention and/or treatment of critical illness, such as treatment of a critically ill patient, a critical illness poly-nephropathy (CIPNP) patient, and/or a potential CIPNP patient; prevention of critical illness or development of CIPNP; prevention, treatment and/or cure of systemic inflammatory response syndrome (SIRS) in a patient; and/or for the prevention or reduction of the likelihood of a patient suffering from bacteraemia, septicaemia, and/or septic shock during hospitalisation; and/or

(xi) prevention and/or treatment of polycystic ovary syndrome (PCOS).

In some embodiments, the indication is selected from the group consisting of (i)-(iii) and (v)-(iix), such as indications (i), (ii), and/or (iii); or indication (v), indication (vi), indication (vii), and/or indication (iix). In some embodiments, the indication is (i). In some embodiments the indication is (v). In some embodiments the indication is (iix). In some embodiments the indications are type 2 diabetes and/or obesity.

In some embodiments, the indication is as an adjunct to diet and exercise to improve glycaemic control in adults with type 2 diabetes. In some embodiments, the indication is reducing the risk of CV death, or reducing the worsening of heart failure in adults with heart failure with reduced ejection fraction (HFrEF) or preserved ejection fraction (HFpEF).

The invention further relates to a method of treatment of an individual in need thereof, comprising administering a therapeutically active amount of a composition according to the present invention to said individual. In some embodiments one or more dose units may be administered to said individual in need.

### Method of Treatment

The invention further relates to a method of treating a subject in need thereof, comprising administering a therapeutically effective amount of a composition according to the present invention to said subject. In some embodiments the method of treatment is for treatment of diabetes or obesity and/or the further indications specified herein.

In some embodiments, a method for treating diabetes is described comprising administering to a subject in need thereof a therapeutically effective amount of a pharmaceutical composition comprising a GLP-1 agonist, a SGLT2 inhibitor, a salt of NAC, a hydrotrope and optionally, a lubricant.

In some embodiments, a method for treating diabetes is described comprising administering to a subject in need thereof a therapeutically effective amount of a pharmaceutical composition comprising

i) 0.1-50 mg semaglutide,

ii) 1-50 mg SGLT2 inhibitor,

iii) 25-600 mg salt of NAC, such as the sodium salt of NAC (SNAC) and

iv) 20-600 mg, such as 50-400 mg, nicotinamide or resorcinol and

v) 0-20 mg lubricant.

In some embodiments, the GLP-1 agonist is semaglutide having a formula of N-epsilon26-[2-(2-{2-[2-(2-{2-[(S)-4-carboxy-4-(17-carboxy-heptadecanoylamino)butyrylamino]ethoxy}ethoxy)acetylamino]ethoxy}ethoxy)acetyl][Aib8,Arg34]GLP-1(7-37) and the salt of NAC is sodium N-(8-(2-hydroxybenzoyl)amino)caprylic acid (SNAC).

Various examples of a lubricant are described, including magnesium stearate. The composition is administered orally and is in a form of a tablet, capsule or a sachet.

In some embodiments one or more dose units may be administered to said subject in need.

### Combination Treatment

The treatment with a composition according to the present invention may also be combined with one or more further active pharmaceutical ingredient(s), e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), Gastric Inhibitory Polypeptides (GIP analogues), compounds lowering food intake, RXR agonists and agents acting on the ATP-dependent potassium channel of the 3-cells; Cholestyramine, colestipol, clofibrate, gemfibrozil, lovastatin, pravastatin, simvastatin, probucol, dextrothyroxine, neteglinide, repaglinide; 3-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, PYY agonists, Y2 receptor agonists, Y4 receptor agonists, mixed Y2/Y4 receptor agonists, MC4 (melanocortin 4) agonists, orexin antagonists, TNF (tumour necrosis factor) agonists, CRF (corticotropin releasing factor) agonists, CRF BP (corticotropin releasing factor binding protein) antagonists, urocortin agonists, β3 agonists, oxyntomodulin and analogues, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists, Gastric Inhibitory Polypeptide agonists or antagonists (GIP analogues), gastrin and gastrin analogues.

Unless otherwise stated, ranges herein include their end points. In some embodiments the term “a” means “one or more”. In some embodiments, and unless otherwise indicated in the specification, terms presented in singular form also include the plural situation. Herein the term “about” means±10% of the value referred to, and includes the value.

The invention as described herein is, without limitation hereto further defined by the embodiments described here below and the claims of the document.

### EMBODIMENTS

1. A composition comprising
   i) a GLP-1 agonist (such as semaglutide),
   ii) 1-50 mg SGLT2 inhibitor,
   iii) a salt of NAC, and
   iv) a hydrotrope.
2. The composition according to embodiment 1, wherein the hydrotrope is selected from the group of hydrotropes consisting of: Nipecotamide, Nicotinamide, p-hydroxybenzoic acid sodium, N,N dimethyl urea, N,N dimethyl benzamide, N,N diethyl nicotinamide, Sodium salicylate, Resorcinol, Sodium benzoate, Sodium Xylenesulfonate, Sodium p-toluenesulfonate, 1-Methylnicotinamide, Pyrogallol, Pyrocathecol, Epigallocatechin gallate, Tannic acid and Gentisic acid sodium salt hydrate.
3. The composition according to embodiment 1, wherein the hydrotrope is selected from the group of hydrotropes consisting of: Nipecotamide, Nicotinamide, p-hydroxybenzoic acid sodium, N,N dimethyl urea, N,N dimethyl benzamide, N,N diethyl nicotinamide, Sodium salicylate, Resorcinol, Sodium Xylenesulfonate, Sodium p-toluenesulfonate, 1-Methylnicotinamide, Pyrogallol, Pyrocathecol, Epigallocatechin gallate, Tannic acid and Gentisic acid sodium salt hydrate.
4. The composition according to any of the preceding embodiments, wherein the hydrotrope comprises an aromatic ring structure.
5. The composition according to any of the preceding embodiments, wherein the hydrotrope is not sodium benzoate.
6. The composition according to any of the preceding embodiments, wherein the hydrotrope has a molecular weight of less than 400 g/mol.
7. The composition according to any of the preceding embodiments, wherein the hydrotrope has a molecular weight of at least 80 g/mol.
8. The composition according to any of the preceding embodiments, wherein the hydrotrope increases the solubility of SNAC at least 2-fold.
9. The composition according to any of one the preceding embodiments, wherein the hydrotrope is increases the solubility of SNAC at least 5-fold.
10. The composition according to any of the preceding embodiments, wherein the hydrotrope is increases the solubility of SNAC at least 10-fold.
11. The composition according to any of embodiments 8-10, wherein the solubility is measured at a concentration of 200 mg/ml of the hydrotrope at pH 6.
12. The composition according to any of embodiments 8-11, where in the solubility is measured as room temperature.
13. The composition according to any of embodiments 8-11, where in the solubility is measured as described in Assay I herein.
14. The composition according to embodiment 1, wherein the hydrotrope is Nicotinamide or Resorcinol.
15. The composition according to embodiment 1, wherein the hydrotrope is Nicotinamide.
16. The composition according to any of the preceding embodiments, wherein the ratio of salt of NAC/hydrotrope (w/w) is at least 0.5.
17. The composition according to any of the preceding embodiments, wherein the ratio of salt of NAC/hydrotrope (w/w) is 0.5-10.0 or such as 0.75-10.0, 0.5-8.0 or 1-2.0.
18. The composition according to any of the preceding embodiments, wherein the ratio of hydrotrope/salt of NAC (w/w) is at least 0.1.
19. The composition according to any of the preceding embodiments, wherein the ratio of hydrotrope/salt of NAC (w/w) is 0.1-5.0 or such as 0.1-4.0, 0.2-3.0 or 0.25-2.0.
20. The composition according to any of the preceding embodiments, wherein the composition comprises a lubricant.
21. The composition according to any of the preceding embodiments, wherein the composition comprises 0.25-5 w/w % lubricant of the total amount of other excipients.
22. The composition according to any of the preceding embodiments, wherein the composition comprises a lubricant selected from magnesium stearate and glyceryl dibehenate.
23. The composition according to any of the preceding embodiments, wherein the composition comprises 0.25-5 w/w % magnesium stearate of the total amount of SNAC and nicotinamide.
24. The composition according to any of the preceding embodiments, wherein a dose unit comprises 0.1-50 mg of the GLP-1 agonist.
25. The composition according to any of the preceding embodiments, wherein the SLGT2 inhibitor is selected from the group consisting of: dapagliflozin, empagliflozin, canagliflozin, ertugliflozin, sotagliflozin, ipragliflozin, tofogliflozin, luseogliflozin, bexagliflozin, and remogloflozin.
26. The composition according to any of the preceding embodiments, wherein the SLGT2 inhibitor is selected from the group consisting of: dapagliflozin and empagliflozin.
27. The composition according to any of the preceding embodiments, wherein the SLGT2 inhibitor is in the form of a pharmaceutically acceptable salt, hydrate and/or solvate thereof.
28. The composition according to any of the preceding embodiments, wherein a dose unit comprises 3-20 mg dapagliflozin, such as 5 mg or 10 mg dapagliflozin.
29. The composition according to any of the preceding embodiments, wherein a dose unit comprises 3-20 mg dapagliflozin, such as 10 mg dapagliflozin.
30. The composition according to any of the preceding embodiments, wherein a dose unit comprises 5-50 mg empagliflozin, such as 25 mg empagliflozin.
31. The composition according to any of the preceding embodiments, wherein the salt of NAC is selected from the group consisting of the sodium salt, potassium salt and/or ammonium salt of NAC.
32. The composition according to any of the preceding embodiments comprising or consisting of:
   i) a GLP-1 agonist (such as semaglutide),
   ii) 1-50 mg SGLT2 inhibitor,
   iii) a salt of NAC, such as the sodium salt of NAC (SNAC),
   iv) nicotinamide or resorcinol, and
   v) a lubricant.
33. The composition according to any of the preceding embodiments comprising or consisting of:
   vi) semaglutide,
   vii) 1-50 mg SGLT2 inhibitor,
   viii) sodium salt of NAC (SNAC),
   ix) nicotinamide or resorcinol, and
   x) a lubricant.
34. The composition according to any of the preceding embodiments, wherein a dose unit comprises at most 1000 mg of said salt of NAC.
35. The composition according to any of the preceding embodiments, wherein a unit dosage comprises
   i) 0.1-50 mg GLP-1 agonist (such as semaglutide),
   ii) 1-50 mg SGLT2 inhibitor,
   iii) 50-600 mg salt of NAC, such as the sodium salt of NAC (SNAC),
   iv) 50-400 mg nicotinamide or resorcinol, and
   v) 0-20 mg lubricant.
36. The composition according to any of the preceding embodiments, wherein a unit dosage comprises
   i) 0.1-50 mg, such as 0.1-25 mg, GLP-1 agonist (such as semaglutide),
   ii) 1-50 mg SGLT2 inhibitor,
   iii) 50-600 mg salt of NAC, such as the sodium salt of NAC (SNAC),
   iv) 50-400 mg nicotinamide or resorcinol, and
   v) 0-20 mg lubricant.
37. The composition according to any of the preceding embodiments, wherein the composition is a solid composition, such as a solid oral dosage form.
38. The composition according to any of the preceding embodiments, wherein the composition comprises a granulate comprising said salt of NAC.
39. The composition according to any of the preceding embodiments, wherein the composition comprises a granulate comprising said hydrotrope (such as nicotinamide or resorcinol), and said lubricant.
40. The composition according to any of the preceding embodiments, wherein the composition comprises a granulate comprising said salt of NAC and said hydrotrope (such as nicotinamide or resorcinol).
41. The composition according to any of the preceding embodiments, wherein said hydrotrope (such as nicotinamide or resorcinol), and said delivery agent are optionally co-processed prior to granulation.
42. The composition according to any of the preceding embodiments, wherein the granulate is obtained by hot melt extrusion and optionally milling, spray granulation, wet granulation or dry granulation.
43. The composition according to any of the preceding embodiments, wherein said salt of NAC and said hydrotrope (such as nicotinamide or resorcinol), are fed into the process stream separately.
44. The composition according to any of the preceding embodiments, wherein said salt of NAC, said hydrotrope (such as nicotinamide or resorcinol), and optionally said SGLT2 inhibitor, are blended prior to granulation.
45. The composition according to any of the preceding embodiments, wherein said salt of NAC and said hydrotrope (such as nicotinamide or resorcinol), said lubricant, and optionally said SGLT2 inhibitor are blended prior to granulation.
46. The composition according to any of the preceding embodiments, wherein said salt of NAC and said hydrotrope (such as nicotinamide or resorcinol), and said lubricant are fed into the process separately.
47. The composition according to any of the preceding embodiments, wherein a solution of said salt of NAC and said hydrotrope (such as nicotinamide or resorcinol) is prepared prior to granulation.
48. The composition according to any of the preceding embodiments, wherein solutions of said salt of NAC, said hydrotrope (such as nicotinamide or resorcinol), and optionally said SGLT2 inhibitor are prepared and fed in the process as separate solutions.
49. The composition according to any of the preceding embodiments, wherein the granulate is obtained by spray granulation or wet granulation.
50. The composition according to any of the preceding embodiments, wherein the solution is spray dried prior to dry granulation.
51. The composition according to any of the preceding embodiments, wherein the granulate is obtained hot melt extrusion and optionally subsequent milling.
52. The composition according to any of the preceding embodiments, wherein the blend of said salt of NAC, said hydrotrope (such as nicotinamide or resorcinol), and optionally said SGLT2 inhibitor is hot melt extruded.
53. The composition according to any of the preceding embodiments, wherein the said salt of NAC and said hydrotrope (such as nicotinamide or resorcinol) are fed in extruder separately prior to hot melt extrusion.
54. The composition according to any of the preceding embodiments, wherein the blend of said salt of NAC and said hydrotrope (such as nicotinamide or resorcinol) and said lubricant are hot melt extruded.
55. The composition according to any of the preceding embodiments, wherein the said salt of NAC and said hydrotrope (such as nicotinamide or resorcinol), and said lubricant are fed in the extruder separately prior to hot melt extrusion.
56. The composition according to any of the preceding embodiments, wherein the extrudate is milled.
57. The composition according to any of the preceding embodiments, wherein the granulate is obtained by roller compaction.
58. The composition according to any of the preceding embodiments, wherein the granulate of co-processed salt of NAC and hydrotrope (such as nicotinamide or resorcinol) is sieved.
59. The composition according to any of the preceding embodiments, wherein the milled hot melt extrudate is sieved through a screen of 50-500 μm.
60. The composition according to any of the preceding embodiments, wherein the spray dried or roller compacted product is sieved through a screen of 50-500 μm.
61. The composition according to any of the preceding embodiments, wherein the granulate is obtained by spray granulation.
62. The composition according to any of the preceding embodiments, wherein a solution of said salt of NAC and said hydrotrope (such as nicotinamide or resorcinol), is processed by spouted bed or fluid bed spray granulation.
63. The composition according to any of the preceding embodiments, wherein solutions of said salt of NAC and said hydrotrope (such as nicotinamide or resorcinol), are prepared and fed in process as separate solutions and being processed by spouted bed or fluid bed spray granulation.
64. The composition according to any of the preceding embodiments, wherein the granulation product is sieved through a 100-500 μm screen.
65. The composition according to any of the preceding embodiments, wherein the composition comprises an extra-granular part.
66. The composition according to any of the preceding embodiments, wherein the granulate is blended with any further excipients and/or pharmaceutically active ingredient(s) prior to compression.
67. The composition according to any of the preceding embodiments, wherein the hydrotrope is included in the intra-granular part.
68. The composition according to any of the preceding embodiments, wherein the GLP-1 agonist is included in an extra granular part.
69. The composition according to any of the preceding embodiments, wherein the GLP-1 agonist and SLGT2 inhibitor are included in an extra-granular part.
70. The composition according to any of the preceding embodiments, wherein the SLGT2 inhibitor is included in an intra-granular part.

The composition according to any of the preceding embodiments, wherein the SLGT2 inhibitor is included in an intra-granular and/or extra-granular part.
71. The composition according to any of the preceding embodiments, wherein the composition is produced by a method comprising the steps of:
   a) granulation of a mixture comprising the delivery agent and the hydrotrope, and optionally the SGLT2 inhibitor, and optionally the lubricant,
   b) blending of the granulates of a) with the GLP-1 agonist, and optionally the SGLT2 inhibitor, and optionally the lubricant,
   c) compressing the blend of b).
72. The composition according to any of the preceding embodiments, wherein the composition is produced by a method comprising the steps of:
   a) granulation of the delivery agent and the hydrotrope, and optionally the SGLT2 inhibitor, and optionally the lubricant,
   b) blending of the granulates of a) with the GLP-1 agonist, and optionally the SGLT2 inhibitor, and optionally the lubricant,
   c) compressing the blend of b).
73. A composition consisting of:
   i) GLP-1 agonist (such as semaglutide),
   ii) 1-50 mg SGLT2 inhibitor,
   iii) a salt of NAC,
   iv) nicotinamide or resorcinol, and
   v) optionally a lubricant, such as magnesium stearate.
74. The composition according to any of the preceding embodiments, wherein said SLGT2 inhibitor is selected from the group consisting of: dapagliflozin, empagliflozin, canagliflozin, ertugliflozin, sotagliflozin, ipragliflozin, tofogliflozin, luseogliflozin, bexagliflozin, and remogloflozin (such as dapagliflozin and empagliflozin).
75. The composition according to any of the preceding embodiments, wherein the salt of NAC is the sodium salt of NAC (SNAC).
76. The composition according to any of the preceding embodiments, wherein the hydrotrope is nicotinamide.
77. The composition according to any of the preceding embodiments, wherein the composition is a pharmaceutical composition.
78. The composition according to any of the preceding embodiments, wherein the composition is for oral administration.
79. The composition according to any of the preceding embodiments, wherein the composition is a solid composition, such as a tablet, a capsule or a sachet for oral administration.
80. The composition according to any one of the preceding embodiments, wherein said GLP-1 agonist comprises GLP-1(7-37) (SEQ ID No: 1) optionally comprising one or more substitutions, deletions, additions and/or modifications.
81. The composition according to any one of the preceding embodiments, wherein said GLP-1 agonist comprises a maximum of 12 amino acids which have been altered, e.g., by substitution, deletion, insertion and/or modification, compared to GLP-1(7-37) (SEQ ID No: 1).
82. The composition according to any one of the preceding embodiments, wherein said GLP-1 agonist comprises a maximum of 10 amino acids which have been altered, e.g., by substitution, deletion, insertion and/or modification, compared to GLP-1(7-37) (SEQ ID No: 1).
83. The composition according to any one of the preceding embodiments, wherein said GLP-1 agonist comprises a maximum of 8 amino acids which have been altered, e.g., by substitution, deletion, insertion and/or modification, compared to GLP-1(7-37) (SEQ ID No: 1).
84. The composition according to any one of the preceding embodiments, wherein said GLP-1 agonist comprises a maximum of 6 amino acids which have been altered, e.g., by substitution, deletion, insertion and/or modification, compared to GLP-1(7-37) (SEQ ID No: 1).
85. The composition according to any one of the preceding embodiments, wherein said GLP-1 agonist comprises one or more substituents.
86. The composition according to any one of the preceding embodiments, wherein said substituent comprises a linker, such as one or more OEG.
87. The composition according to any one of the preceding embodiments, wherein said linker is located proximally in said substituent to the point of attachment between said substituent and the peptide in said GLP-1 agonist.
88. The composition according to any one of the preceding embodiments, wherein said GLP-1 agonist has an EC50 (without HSA) of at most 500 pM.
89. The composition according to any one of the preceding embodiments, wherein said GLP-1 agonist is selected from the group consisting of semaglutide, Compound A and Compound B.
90. The composition according to any one of the preceding embodiments, wherein said GLP-1 agonist is semaglutide.
91. The composition according to any one of the preceding embodiments, wherein said GLP-1 agonist is Compound A.
92. The composition according to any one of the preceding embodiments, wherein said GLP-1 agonist is Compound B.
93. The composition according to any of the preceding embodiments, wherein the composition is a pharmaceutical composition for use in a method of treating diabetes and/or obesity.
94. A method for producing a solid pharmaceutical composition comprising the steps of:
   i) co-processing a salt of NAC and a hydrotrope (and optionally an SGLT2 inhibitor and/or a lubricant), and
   ii) preparing said solid pharmaceutical composition using the product of i),
   wherein said solid pharmaceutical composition comprises a GLP-1 agonist (such as semaglutide) and an SGLT2 inhibitor and optionally a lubricant.
95. A method for producing a solid pharmaceutical composition comprising the steps of:
   i) obtaining a blend comprising a salt of NAC and a hydrotrope (and optionally a SGLT2 inhibitor and/or a lubricant), or feeding the ingredients separately and
   ii) co-processing the blend or ingredients of i), and
   iii) preparing said solid pharmaceutical composition using the product of ii),
   wherein said solid pharmaceutical composition also comprises GLP-1 agonist (such as semaglutide) and an SGLT2 inhibitor and optionally a lubricant.
96. A method for producing a solid pharmaceutical composition comprising the steps of:
   i) feeding a salt of NAC and a hydrotrope (and/or optionally a SGLT2 inhibitor and/or a lubricant in the process, and
   ii) co-processing the ingredients of i), and
   iii) preparing said solid pharmaceutical composition using the product of ii),
   wherein said solid pharmaceutical composition also comprises GLP-1 agonist (such as semaglutide) and an SGLT2 inhibitor and optionally a lubricant.
97. A method for producing a solid pharmaceutical composition according to any of the preceding embodiments, wherein said co-processing step is performed by
   a) hot melt extrusion
   b) spray-granulation
   c) dry granulation or
   d) spray-drying.
98. A method for producing a solid pharmaceutical composition comprising the steps of:
   i) obtaining a blend comprising a salt of NAC and a hydrotrope, (and optionally a SGLT2 inhibitor and/or a lubricant) or feeding the ingredients separately, and
   ii) hot melt extruding the blend or ingredients of i)
   iii) optionally milling the extrudate of ii) and
   iv) preparing said solid pharmaceutical composition using the product of iii),
      wherein said solid pharmaceutical composition comprises GLP-1 agonist (such as semaglutide) and an SGLT2 inhibitor and/or a lubricant.
99. A method for producing a solid pharmaceutical composition comprising the steps of
   i) obtaining a blend comprising a salt of NAC and a hydrotrope, or feeding the ingredients separately, and
   ii) co-processing the blend or the ingredients of i),
   iii) admixing the product of ii) with active pharmaceutical ingredients and optionally any further excipients and
   iv) preparing said solid pharmaceutical composition using the mixture of iii),
      wherein said solid pharmaceutical composition comprises GLP-1 agonist (such as semaglutide) and an SGLT2 inhibitor.
100. A method for producing a solid pharmaceutical composition comprising the steps of
   i) obtaining a blend comprising a salt of NAC and a hydrotrope, or feeding the ingredients separately, and
   ii) hot melt extruding the blend or the ingredients of i)
   iii) milling the extrudate of ii)
   iv) admixing the product of iii) with active pharmaceutical ingredients and optionally any further excipients and
   v) preparing said solid pharmaceutical composition using the mixture of iv),
      wherein said solid pharmaceutical composition comprises GLP-1 agonist (such as semaglutide) and an SGLT2 inhibitor.
101. A method for producing a composition according to any of embodiments 93-99, wherein the pharmaceutical composition is defined as in any of embodiments 1-92.
102. A method for treatment of diabetes or obesity comprising administering to a subject in need a therapeutically effective amount of a composition as defined in any one of embodiments 1-92 or a composition produced by the method as defined in any one of embodiments 93-100.
103. The method according to embodiment 100, wherein said composition is administered, once daily or less frequent.

### EXAMPLES

### Materials and Methods

Assay I: SNAC Solubility in Combination with Selected Hydrotropes

Hydrotropes are weighed off and dissolved in 5 mL ultrapure water (200 mg/mL) and pH was titrated to pH 6 by addition of 2M HCl. Subsequently SNAC (200 mg) is added to the samples and placed on magnetic stirrers (400 rpm). The pH is maintained at pH 6 throughout the experiment by addition of 2M HCl.

After 4 hours of incubation at room temperature the samples were filtered through 0.45 μm syringe filters and the concentration of SNAC in solution is determined using a RP-HPLC method for detection of SNAC. The sample content is calculated based on the peak area of the SNAC peak in the chromatogram relative to the peak area of the SNAC references.

### Assay II: SNAC Solubility in Varying Concentrations of Nicotinamide and Resorcinol

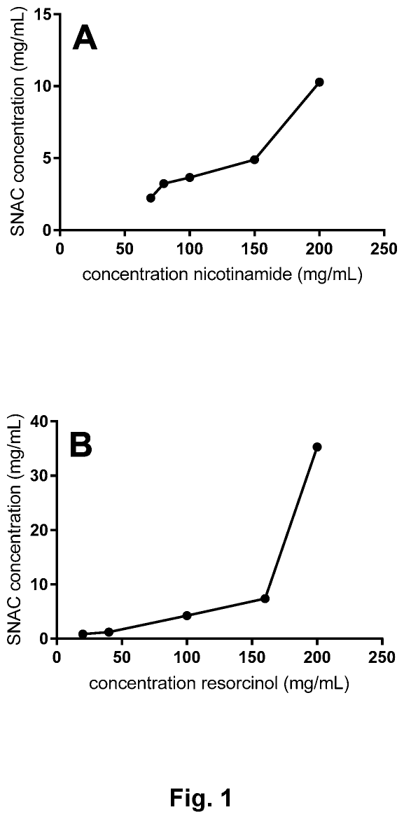

Nicotinamide or resorcinol was weighed off and dissolved in 5 mL ultrapure water to the final concentrations shown in FIGS. 1A and 1B and pH was titrated to pH 6 by addition of 2M HCl. Subsequently SNAC (200 mg) was added to the samples placed on magnetic stirrers (400 rpm) and pH is maintained at pH 6 throughout the experiment by addition of 2M HCl.

After 4 hours of incubation samples were filtered through 0.45 μm syringe filters and the concentration of SNAC in solution is determined using a RP-HPLC method for detection of SNAC. The sample content is calculated based on the peak area of the SNAC peak in the chromatogram relative to the peak area of the SNAC references. The results are shown in FIG. 1 demonstrating a concentration dependent effect on SNAC solubility of both hydrotropes.

### Assay III: Dissolution Test

A dissolution test is performed in an appropriate dissolution apparatus e.g. USP dissolution apparatus 2, and a standard dissolution test according to the European Pharmacopeia (Ph Eur 2.9.3) may be performed to measure the release of the GLP-1 agonist, SGLT2 inhibitor and SNAC in vitro.

Data described herein is obtained using apparatus 2 in accordance with United States Pharmacopoeia 35 using a paddle rotation speed of 50 rpm (Reference D and compositions D1-D3) or apparatus 1 in accordance with United States Pharmacopoeia 35 using a basket rotation of 75 rpm (compositions D4 and D5). For testing at pH 6.8, the 500 mL dissolution medium of 0.05 M phosphate buffer is used at a temperature of 37±0.5° C. Dissolution media has a content of 0.1% Brij35. Sampling was done at appropriate intervals. Sample content is determined using a RP-UHPLC method for triple detection of GLP-1 agonist, SGLT2 inhibitor and SNAC. The sample content is calculated based on the peak area of the GLP-1 agonist, SGLT2 inhibitor and SNAC peaks in the chromatogram relative to the peak areas of the GLP-1 agonist, SGLT2 inhibitor and SNAC references, respectively. The released amount of GLP-1 agonist, SGLT2 inhibitor and SNAC is calculated as percentages of the actual content in the tablets i.e. 100/300 mg/tablet SNAC and 3/14 mg/tablet GLP-1 agonist (e.g. semaglutide) and 10 mg/tablet SGLT2 inhibitor. The actual content in the tablets is determined using Assay IV. The cumulative released amount of GLP-1 agonist and SGLT2 inhibitor may be reported as average of 3 tablets and data presented herein is normalised to the highest value.

### Assay IV: Analysis of Amount of GLP-1 Agonist, SGLT2 Inhibitor and SNAC

The GLP-1 agonist, SGLT2 inhibitor and SNAC are extracted from the tablets. Tablets are dissolved in a relevant amount of 0.05 M phosphate buffer, pH 7.4, with 20% acetonitrile. Extraction time of two hours is used, and samples are centrifuged before analysis. Standards of relevant GLP-1 agonist, SGLT2 inhibitor and SNAC are prepared by using the same diluent as for the samples. UHPLC with an UV-detector or fluorescence-detector is used for determining the GLP-1 agonist, SGLT2 inhibitor and SNAC content. The sample content is calculated based on the peak area of the GLP-1 agonist, SGLT2 inhibitor and SNAC peaks in the chromatogram relative to the peak areas of the GLP-1 agonist, SGLT2 inhibitor and SNAC references, respectively.

### General Methods for Tablet Preparation

### Method 1: Hot Melt Extrusion

Hot melt extrusion is carried out on a Thermo Scientific Process 11 twin screw extruder. SNAC and nicotinamide or resorcinol are blended on a Turbula mixer prior to feeding into the extruder (7 min 25 rpm). The equipment is operated at process temperatures varying between 200° C. to 105° C. along the barrel to facilitate the melt extrusion. The screw speed is varied between 50-1000 rpm and material is fed into the extruder using a gravimetric feeder at varying feed rates and extruded through a 2 mm diameter circular die. The resulting extrudates are manually sieved into granules using a final mesh screen between 355 and 150 μm.

### Method 2: Blending for Tablet Compression

Blending is carried out by manual geometric mixing the intermediate granulate with GLP-1 agonist and SGLT2 inhibitor followed by blending on a Turbula mixer (7 min, 25 rpm). In compositions including magnesium stearate or glyceryl dibehenate it was sieved through a 125 μm or 355 μm mesh and added in a secondary blending step by manual geometric mixing followed by blending on a Turbula mixer (2 min, 25 rpm).

### Method 3: Tablet Preparation

Tablets are produced on a Kilian Style One (may be referred to as Kilian Styl'One) or a Fette 102I mounted with a single set of punches, resulting in 5.75 mm×10 mm, 7.5 mm×13 mm or 8.5 mm×16 mm oval compound cup tablets having no score. Punch size is chosen according to the total tablet weight. For the Kilian Style One the press speed is set to 10% or 20 rpm and for Fette 102i the press speed is set at 20 rpm. The fill volume is adjusted to obtain tablets having target weights based on composition. Compression forces around 3 to 25 kN are applied to obtain tablets with a crushing strength of around 20-120 N respective to the tablet size.

### Example 1—SNAC Solubility in Combination with Selected Hydrotropes

A series of 18 different hydrotropes were selected for testing according to Assay I. Results obtained are presented in Table 1, demonstrating that the majority of hydrotropes increase solubility of SNAC significantly.

TABLE 1
Hydrotropic effect of selected hydrotropes (200 mg/mL) on SNAC
solubility at pH 6

| | | SNAC | |
|---|---|---|---|
| | Replicates | concentration | Fold increase |
| Hydrotrope | (n) | (mg/mL) | in solubility |
| SNAC control (no hydrotrope) | 1 | 0.59 | 1 |
| Nipecotamide | 1 | 1.55 | 2.6 |
| Nicotinamide | 5 | 8.63 | 14.6 |
| p-hydroxybenzoic acid sodium | 1 | 1.25 | 2.1 |
| N,N dimethyl urea | 1 | 2.34 | 4.0 |
| N,N dimethyl benzamide | 4 | 22.46 | 38 |
| N,N diethyl nicotinamide | 1 | 4.58 | 7.8 |
| Sodium salicylate | 1 | 1.59 | 2.7 |
| Resorcinol | 6 | 29.95 | 51 |
| Sodium benzoate | 2 | 1.25 | 2.1 |
| Urea | 1 | 0.77 | 1.3 |
| Sodium Xylenesulfonate | 1 | 2.01 | 3.4 |
| Sodium p-toluenesulfonate | 1 | 1.78 | 3.0 |
| 1-Methylnicotinamide | 1 | 2.20 | 3.7 |
| Pyrogallol | 4 | 8.98 | 15 |
| Pyrocathecol | 1 | 23.95 | 40.6 |
| Epigallocatechin gallate | 1 | 5.66 | 9.6 |
| Tannic acid | 1 | 6.31 | 10.7 |
| Gentisic acid sodium salt hydrate | 1 | 2.72 | 4.6 |

### Example 2—Solid Compositions Comprising Semaglutide and an SGLT2 Inhibitor

Tablets with different amounts of the GLP-1 agonist semaglutide, SGLT2 inhibitor, SNAC and further excipients were prepared. The content of the prepared compositions is provided in Table 2. SGLT2 inhibitor A is dapagliflozin propylene glycol hydrate. Compositions 01-03 and D4-D5 were generally prepared as described in Method 1 and Method 2 and Method 3 above. Composition Reference 0 was generally prepared as described in WO2013/139694. The SGLT2 inhibitor was added extra-granular.

TABLE 2
Tablet compositions expressed as mg per tablet

| | | SGLT2 | | | Magnesium | | |
|---|---|---|---|---|---|---|---|
| | Semaglutide | inhibitor | SNAC | Nicotinamide | stearate | Povidone | MCC |
| Composition | (mg) | A (mg) | (mg) | (mg) | (mg) | (mg) | (mg) |
| Reference D | 5 | 5 | 300 | — | 9.7 | 8 | 80 |
| D1 | 14 | 10 | 100 | 66.67 | 1.97 | — | — |
| D2 | 14 | 10 | 300 | 200 | 5.36 | — | — |
| D3 | 3 | 10 | 100 | 66.67 | 1.84 | — | — |
| D4 | 3 | 10 | 100 | 66.7 | 0.83 | — | — |
| D5 | 14 | 10 | 300 | 200 | 2.5 | — | — |

### Example 3—Dissolution Testing

The objective of the present study was to evaluate the dissolution of the compositions described in Example 2.

Dissolution was measured according to Assay III and the amount of the GLP-1 agonist and SGLT2 inhibitor and SNAC were measured according to Assay IV. The released amount of GLP-1 agonist and SGLT2 inhibitor and SNAC were calculated as percentages of the actual content in the test compositions i.e. 100/300 mg/tablet of SNAC and 10 mg/tablet of SGLT2 inhibitor and 3/14 mg/tablet GLP-1 agonist. The amount of GLP-1 agonist and SGLT2 inhibitor released is reported as average of 3 tablets.

Table 3 and 4 show the results for test compositions prepared according to Example 1 above, wherein the release is presented as “GLP-1 agonist in solution (%)” and “SGLT2 inhibitor in solution (%)” describing the amount of GLP-1 agonist and SGLT2 inhibitor in solution after 15, 30 and 60 min relative to the total amount of GLP-1 agonist and SGLT2 inhibitor in the tablet at the start of the experiment. The data presented is normalised to the highest value. The total content of GLP-1 agonist and SGLT2 inhibitor and SNAC in the tablets were determined according to Assay IV.

TABLE 3
GLP-1 agonist in solution (%)

| | GLP-1 agonist in solution (%) | | |
|---|---|---|---|
| Composition | 15 min | 30 min | 60 min |
| Reference D | 43.8 | 69.9 | Full release |
| D1 | Full release* | Full release | Full release |
| D2 | Full release | Full release | Full release |
| D3 | Full release | Full release | Full release |
| D4 | Full release | Full release | Full release |
| D5 | Full release | Full release | Full release |

*Full release is defined as >95% of the compound has been released

TABLE 4
SGLT2 inhibitor in solution (%)

| | SGLT2 inhibitor in solution (%) | | |
|---|---|---|---|
| Composition | 15 min | 30 min | 60 min |
| Reference D | 46.5 | 71.2 | Full release |
| D1 | Full release* | Full release | Full release |
| D2 | Full release | Full release | Full release |
| D3 | Full release | Full release | Full release |
| D4 | Full release | Full release | Full release |
| D5 | Full release | Full release | Full release |

*Full release is defined as >95% of the compound has been released

The data obtained for Table 3 and Table 4 normalised against the highest value within 70 minutes of the dissolution test according to Assay III for Reference 0 is shown in Table 5 while for compositions 01-05 all results for all time points remained unchanged from Table 3 and Table 4, i.e. “Full release”.

TABLE 5
API in solution (%) for Reference D

| | API in solution (%) | | |
|---|---|---|---|
| API | 15 min | 30 min | 60 min |
| GLP-1 agonist | 35.3 | 56.4 | 80.7 |
| SGLT2 inhibitor | 36.8 | 56.3 | 79.0 |

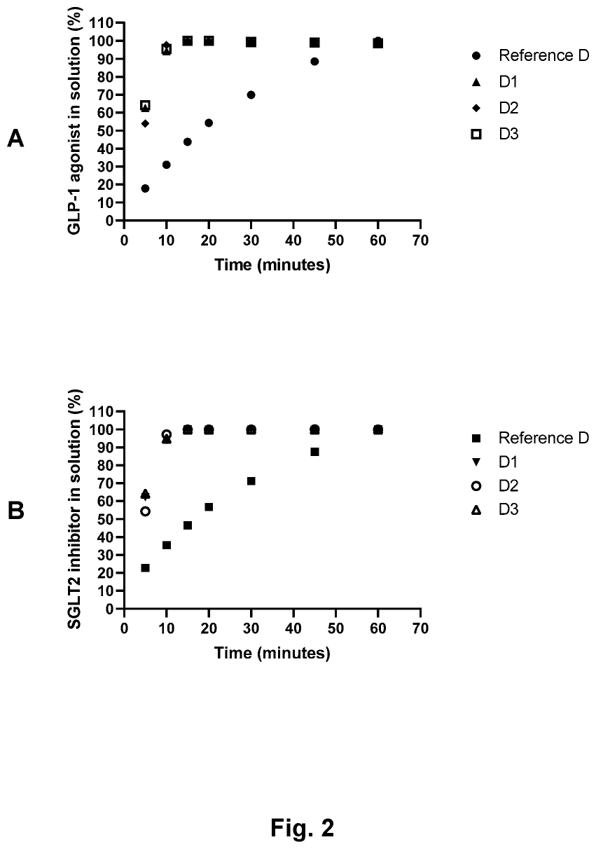

The results obtained show that test compositions D1-D3 and D4-D5 display a faster release of the GLP-1 agonist and SGLT2 inhibitor compared to what was observed for reference D. A significantly faster release of the GLP-1 agonist and SGLT2 inhibitor was observed for the early time points, i.e. at 15 and 30 minutes. The difference in release was not significant after 60 minutes. The amount of SNAC in the test compositions did not influence the release of the GLP-1 agonist and SGLT2 inhibitor after 15 min, i.e. test compositions comprising 100 mg SNAC dissolve as fast as test compositions comprising 300 mg SNAC. Further data obtained after 5, 10, 15, 20, 30, 45 and 60 min for test compositions D1 to D3 are shown in FIG. 2, demonstrating that test compositions D1-D3 are superior to reference D up to the 50 min time point.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising:
i) semaglutide,
ii) 1-50 mg of a sodium-glucose linked transporter-2 (SLGT2) inhibitor,
iii) a salt of N-(8-(2-hydroxybenzoyl)amino)caprylic acid (NAC), and
iv) a hydrotrope,
wherein the SGLT2 inhibitor is selected from the group consisting of dapagliflozin, empagliflozin, canagliflozin, ertugliflozin, sotagliflozin, ipragliflozin, tofogliflozin, luseogliflozin, bexagliflozin, and remogloflozin; and
wherein the hydrotrope increases the solubility of SNAC at least 5-fold or at least 10-fold.

2. The composition according to claim 1, wherein the hydrotrope is selected from nicotinamide and resorcinol.

3. The composition according to claim 1 consisting of:
i) semaglutide,
ii) 1-50 mg SLGT2 inhibitor,
iii) a salt of NAC,
iv) nicotinamide or resorcinol, and
v) a lubricant.

4. The composition according to claim 1, wherein a unit dosage comprises:
i) 0.1-50 mg semaglutide,
ii) 1-50 mg SLGT2 inhibitor,
iii) 50-600 mg salt of NAC,
iv) 20-400 mg nicotinamide, and
v) 0-20 mg lubricant.

5. The composition according to claim 1, wherein said composition or unit dosage comprises 0.1-50 mg or 0.1-25 mg semaglutide.

6. The composition according to claim 1, wherein said unit dosage comprises 5 mg dapagliflozin, 10 mg dapagliflozin or 25 mg empagliflozin.

7. The composition according to claim 1, wherein the ratio of salt of NAC/hydrotrope (w/w) is 0.5-10, such as 0.5-8 or such as 0.5-5.

8. The composition according to claim 1, wherein the composition further comprises a lubricant selected from magnesium stearate and/or glyceryl dibehenate.

9. The composition according to claim 1, wherein the salt of NAC is the sodium salt of N-(8-(2-hydroxybenzoyl)amino)caprylic acid (SNAC).

10. The composition according to claim 1, wherein the composition is a solid composition.

11. The composition according to claim 1, wherein the composition is a pharmaceutical composition for oral administration.

12. A method of treating diabetes and/or obesity comprising administering to a subject in need of such method an amount of a composition according to claim 1.

13. A method for producing a solid pharmaceutical composition comprising the steps of:
i) co-processing a salt of NAC, a hydrotrope, and optionally an SGLT2 inhibitor and/or a lubricant, and
ii) preparing said solid pharmaceutical composition using the product of i),
wherein said solid pharmaceutical composition comprises the GLP-1 agonist semaglutide and an SGLT2 inhibitor and/or a lubricant.

14. A method for producing a solid pharmaceutical composition comprising the steps of:
i) hot melt extruding a salt of NAC and a hydrotrope, (and optionally an SGLT2 inhibitor and/or a lubricant),
ii) optionally milling the extrudate of i) and
iii) preparing said solid pharmaceutical composition using the product of ii),
wherein said solid pharmaceutical composition comprises the GLP-1 agonist semaglutide and an SGLT2 inhibitor and/or a lubricant.

15. A method for producing a solid pharmaceutical composition comprising the steps of
i) hot melt extruding a salt of NAC and a hydrotrope, and optionally an SGLT2 inhibitor and/or a lubricant,
ii) admixing the extrudate of i) with one or more active pharmaceutical ingredients and optionally any further excipients, and
iii) preparing said solid pharmaceutical composition using the mixture of ii),
wherein said solid pharmaceutical composition comprises the GLP-1 agonist semaglutide and an SGLT2 inhibitor and/or a lubricant.

16. The composition according to claim 1, wherein the SGLT2 is selected from dapagliflozin and empagliflozin.


SEQUENCE LISTING (2 sequences)

SEQ ID NO: 1 - protein, 31 aa, Homo sapiens
HAEGTFTSDVSSYLEGQAAKEFIAWLVKGRG

SEQ ID NO: 2 - protein, 31 aa, Artificial Sequence
  GLP-1 receptor agonist
  MOD_RES (2)..(2): Aib
  MOD_RES (20)..(20): This residue is covalently attached to a substituent
HXEGTFTSDVSSYLEGQAAKEFIAWLVRGRG